SEQUENCE LISTING (7 sequences)

SEQ ID NO: 1 - DNA, 19 bp, Artificial sequence
  Synthetic oligonucleotide
  misc_feature (3)..(3): RNA
  misc_feature (10)..(10): RNA
  misc_feature (13)..(13): RNA
  misc_feature (17)..(17): RNA
tauacaaccuacuaccuca

SEQ ID NO: 2 - DNA, 18 bp, Artificial sequence
  Synthetic oligonucleotide
  misc_feature (10)..(10): RNA
acaccattgucacactcc

SEQ ID NO: 3 - DNA, 16 bp, Artificial sequence
  Synthetic oligonucleotide
  misc_feature (5)..(5): RNA
  misc_feature (8)..(8): RNA
accautgucacactcc

SEQ ID NO: 4 - DNA, 14 bp, Artificial sequence
  Synthetic oligonucleotide
  misc_feature (6)..(6): RNA
cattgucacactcc

SEQ ID NO: 5 - DNA, 13 bp, Artificial sequence
  Synthetic oligonucleotide
  misc_feature (5)..(5): RNA
attgucacactcc

SEQ ID NO: 6 - DNA, 12 bp, Artificial sequence
  Synthetic oligonucleotide
  misc_feature (4)..(4): RNA
ttgucacactcc

SEQ ID NO: 7 - DNA, 18 bp, Artificial sequence
  Synthetic oligonucleotide
  misc_feature (10)..(10): RNA
aacaccttgucacactcc

(19) United States
(12) Patent Application Publication
(10) Pub. No.: US 2022/0098581 A1
(43) Pub. Date: Mar. 31, 2022

(54) Methods for Oral Delivery of Oligonucleotides

(71) Applicant: Regulus Therapeutics Inc., San Diego, CA (US)

(72) Inventor: Timothy Wright, San Diego, CA (US)

(73) Assignee: Regulus Therapeutics Inc., San Diego, CA (US)

(21) Appl. No.: 17/259,366

(22) PCT Filed: Jul. 19, 2019
(86) PCT No.: PCT/US2019/042561

§ 371 Date: Jan. 11, 2021

Related U.S. Application Data
(60) Provisional application No. 62/701,317, filed on Jul. 20, 2018.

(51) Int. Cl. C12N 15/113

(52) U.S. Cl. CPC C12N 15/113; C12N 2310/113; C12N 2310/3515; C12N 2320/35; C12N 2310/315; C12N 2310/351; C12N 2310/3231

(57) ABSTRACT

Provided herein are methods for oral administration of oligonucleotides. Further provided herein are methods for oral administration of modified oligonucleotides targeted to microRNA.

### CROSS-REFERENCE TO RELATED APPLICATIONS

This application claims the benefit of priority of U.S. Provisional Application No. 62/701,317, filed Jul. 20, 2018, which is incorporated by reference herein in its entirety for any purpose.

### FIELD OF INVENTION

Provided herein are methods for the oral administration of oligonucleotides.

### DESCRIPTION OF RELATED ART

Currently, the preferred route of administration for oligonucleotides, including anti-miR oligonucleotides, is through subcutaneous injection. As oral administration may be more convenient, some studies of oral administration of oligonucleotides have been performed. For example, oral administration of a modified oligonucleotide formulated with a permeation enhancer resulted in an average bioavailability of 9.5% in healthy volunteers, relative to subcutaneous injection (Tillman et al., 2008, J. Pharm. Sci., 97(1):225-36). Despite initially positive results, challenges for oral delivery of oligonucleotides remain and compounds acting through antisense mechanisms have not advanced into development for the treatment of disease.

### SUMMARY OF INVENTION

Embodiment 1. A method of inhibiting the activity of a target RNA, comprising administering to a subject a compound comprising a modified oligonucleotide complementary to the target RNA, or a pharmaceutically acceptable salt thereof, wherein the modified oligonucleotide has a length of 6 to 25 linked nucleotides and wherein the administration is oral administration.
Embodiment 2. The method of embodiment 1, wherein the target RNA is a microRNA.
Embodiment 3. The method of embodiment 1, wherein the target RNA is a pre-messenger RNA.
Embodiment 4. The method of embodiment 1, wherein the target RNA is a messenger RNA.
Embodiment 5. The method of embodiment 1, wherein the target RNA is a long non-coding RNA.
Embodiment 6. The method of any one of embodiments 1 to 5, wherein the target RNA is expressed in the kidney and the compound consists of the modified oligonucleotide.
Embodiment 7. The method of any one of embodiments 1 to 5, wherein the target RNA is expressed in the liver and the compound comprises the modified oligonucleotide linked to a conjugate moiety.
Embodiment 8. The method of any one of embodiments 1 to 7, wherein the subject has a disease mediated by the target RNA.
Embodiment 9. The method of embodiment 8, wherein the oral administration of the compound improves one or more symptoms of the disease.
Embodiment 10. A method of inhibiting the activity of a microRNA, comprising administering to a subject a compound comprising a modified oligonucleotide complementary to the microRNA, or a pharmaceutically acceptable salt thereof, wherein the modified oligonucleotide has a length of 6 to 25 linked nucleotides and wherein the administration is oral administration.
Embodiment 11. The method of embodiment 1, wherein the modified oligonucleotide is fully complementary to the microRNA.
Embodiment 12. The method of embodiment 1 or 2, wherein the microRNA is expressed in the kidney and the compound consists of the modified oligonucleotide.
Embodiment 13. The method of any one of embodiments 1 to 3, wherein the microRNA is expressed in the liver and the compound comprises the modified oligonucleotide linked to a conjugate moiety.
Embodiment 14. The method of any one of embodiments 1 to 4, wherein the modified oligonucleotide has a length of 6 to 21 linked nucleosides.
Embodiment 15. The method of any one of embodiments 1 to 4, wherein the modified oligonucleotide has a length of 6 to 18 linked nucleosides.
Embodiment 16. The method of any one of embodiments 1 to 4, wherein the modified oligonucleotide has a length of 6 to 15 linked nucleosides.
Embodiment 17. The method of any one of embodiments 1 to 4, wherein the modified oligonucleotide has a length of 6 to 12 linked nucleosides.
Embodiment 18. The method of any one of embodiments 1 to 4, wherein the modified oligonucleotide has a length of 8 to 12 linked nucleosides.
Embodiment 19. The method of any one of embodiments 1 to 4, wherein the modified oligonucleotide has a length of 8 to 13 linked nucleosides.
Embodiment 20. The method of any one of embodiments 1 to 4, wherein the modified oligonucleotide is 6 linked nucleosides in length.
Embodiment 21. The method of any one of embodiments 1 to 4, wherein the modified oligonucleotide is 7 linked nucleosides in length.
Embodiment 22. The method of any one of embodiments 1 to 4, wherein the modified oligonucleotide is 8 linked nucleosides in length.
Embodiment 23. The method of any one of embodiments 1 to 4, wherein the modified oligonucleotide is 9 linked nucleosides in length.
Embodiment 24 The method of any one of embodiments 1 to 4, wherein the modified oligonucleotide is 10 linked nucleosides in length.
Embodiment 25. The method of any one of embodiments 1 to 4, wherein the modified oligonucleotide is 11 linked nucleosides in length.
Embodiment 26. The method of any one of embodiments 1 to 4, wherein the modified oligonucleotide is 12 linked nucleosides in length.
Embodiment 27. The method of any one of embodiments 1 to 13, wherein the modified oligonucleotide is 13 linked nucleosides in length.
Embodiment 28. The method of any one of embodiments 1 to 27, wherein the modified oligonucleotide comprises at least one nucleoside with a modified sugar moiety.
Embodiment 29. The method of embodiment 28, wherein each nucleoside of the modified oligonucleotide comprises a modified sugar moiety.
Embodiment 30. The method of embodiment 28 or 29, wherein each modified sugar moiety is independently selected from a 2′-O-methyl sugar moiety, a 2′-O-methoxyethyl sugar moiety, a 2′-fluoro sugar moiety, and a bicyclic sugar moiety.
Embodiment 31. The method of embodiment 30, wherein each bicyclic sugar moiety is independently selected from a cEt sugar moiety and an LNA sugar moiety.
Embodiment 32. The method of embodiment 31, wherein the cEt nucleoside is an S-cEt nucleoside.
Embodiment 33. The method of any of embodiments 1 to 27, wherein the modified oligonucleotide comprises a plurality of non-bicyclic nucleosides and a plurality of bicyclic nucleosides.
Embodiment 34. The method of embodiment 33, wherein each non-bicyclic nucleoside is independently selected from a 2′-O-methyl nucleoside, a 2′-O-methoxyethyl nucleoside, and a 2′-fluoronucleoside.
Embodiment 35. The method of embodiment 34, wherein each bicyclic nucleoside is selected from a cEt nucleoside and an LNA nucleoside.
Embodiment 36. The method of embodiment 35, wherein the cEt nucleoside is an S-cEt nucleoside.
Embodiment 37. The method of any one of embodiments 1 to 36, wherein the modified oligonucleotide comprises at least one modified internucleoside linkage.
Embodiment 38. The method of any one of embodiments 1 to 36, wherein each internucleoside linkage of the modified oligonucleotide is a modified internucleoside linkage.
Embodiment 39. The method of embodiment 37 or 38, wherein the modified internucleoside linkage is a phosphorothioate linkage.
Embodiment 40. The method of embodiment 7 or 13, wherein the conjugate moiety comprises a cholesterol moiety or a carbohydrate moiety.
Embodiment 41. The method of embodiment 40, wherein the carbohydrate moiety is selected from is selected from N-acetylgalactosamine, galactose, galactosamine, N-formylgalactosamine, N-propionyl-galactosamine, N-n-butanoylgalactosamine, and N-iso-butanoyl-galactosamine.
Embodiment 42. The method of embodiment 7 or 13, wherein the compound has the structure:

Lₙ-linker-X₁—Nₘ-X₂-MO;
wherein each L is, independently, a ligand and n is from 1 to 10; each N is, independently, a modified or unmodified nucleoside and m is from 1 to 5; X₁ is a phosphodiester linkage or a phosphorothioate linkage; X₂ is a phosphodiester linkage or a phosphorothioate linkage; and MO is the modified oligonucleotide.
Embodiment 43. The method of embodiment 42, wherein if n is greater than 1, Ln-linker has the structure:

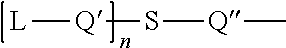

wherein each L is, independently, a ligand; n is from 1 to 10; S is a scaffold; and Q′ and Q″ are, independently, linking groups.
Embodiment 44. The method of embodiment 43, wherein Q′ and Q″ are each independently selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid.
Embodiment 45 The method of embodiment 43 or 44, wherein the scaffold links 2, 3, 4, or 5 ligands to a modified oligonucleotide.
Embodiment 46. The method of embodiment 43 or 44, wherein the scaffold links 3 ligands to a modified oligonucleotide.
Embodiment 47. The method of any one of embodiments 42 to 46 comprising the structure:

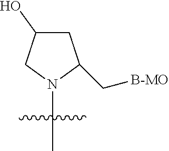

wherein:
B is selected from —O—, —S—, —N(R^{N})—, —Z—P(Z′)(Z″)O—, —Z—P(Z′)(Z″)O—Nₘ—X₁—, and —Z—P(Z′)(Z″)O—Nₘ—X₂—;
MO is the modified oligonucleotide;
R^{N} is selected from H, methyl, ethyl, propyl, isopropyl, butyl, and benzyl;
Z, Z′, and Z″ are each independently selected from O and S;
each N is, independently, a modified or unmodified nucleoside;
m is from 1 to 5;
X₁ is selected from a phosphodiester linkage and a phosphorothioate linkage;
X₂ is a phosphodiester linkage; and
the wavy line indicates the connection to the rest of the linker and ligand(s).
Embodiment 48. The method of any one of embodiments 42 to 47, wherein n is from 1 to 5, 1 to 4, 1 to 3, or 1 to 2.
Embodiment 49. The method of any one of embodiments 42 to 48, wherein n is 3.
Embodiment 50. The method of any one of embodiments 42 to 49, wherein at least one ligand is selected from a carbohydrate, cholesterol, a lipid, a phospholipid, an antibody, a lipoprotein, a hormone, a peptide, a vitamin, a steroid, and a cationic lipid.
Embodiment 51. The method of any one of embodiments 42 to 50, wherein at least one ligand is selected from mannose, glucose, galactose, ribose, arabinose, fructose, fucose, xylose, D-mannose, L-mannose, D-galactose, L-galactose, D-glucose, L-glucose, D-ribose, L-ribose, D-arabinose, L-arabinose, D-fructose, L-fructose, D-fucose, L-fucose, D-xylose, L-xylose, alpha-D-mannofuranose, beta-D-mannofuranose, alpha-D-mannopyranose, beta-D-mannopyranose, alpha-D-glucofuranose, Beta-D-glucofuranose, alpha-D-glucopyranose, beta-D-glucopyranose, alpha-D-galactofuranose, beta-D-galactofuranose, alpha-D-galactopyranose, beta-D-galactopyranose, alpha-D-ribofuranose, beta-D-ribofuranose, alpha-D-ribopyranose, beta-D-ribopyranose, alpha-D-fructofuranose, alpha-D-fructopyranose, glucosamine, galactosamine, sialic acid, and N-acetylgalactosamine.
Embodiment 52. The method of any one of embodiments 42 to 50, wherein at least one ligand is selected from N-acetylgalactosamine, galactose, galactosamine, N-formylgalactosamine, N-propionyl-galactosamine, N-n-butanoylgalactosamine, and N-iso-butanoyl-galactosamine.
Embodiment 53. The method of any one of embodiments 42 to 50, wherein each ligand is N-acetylgalactosamine.
Embodiment 54. The method of any one of embodiments 42 to 50, wherein the compound has the structure:

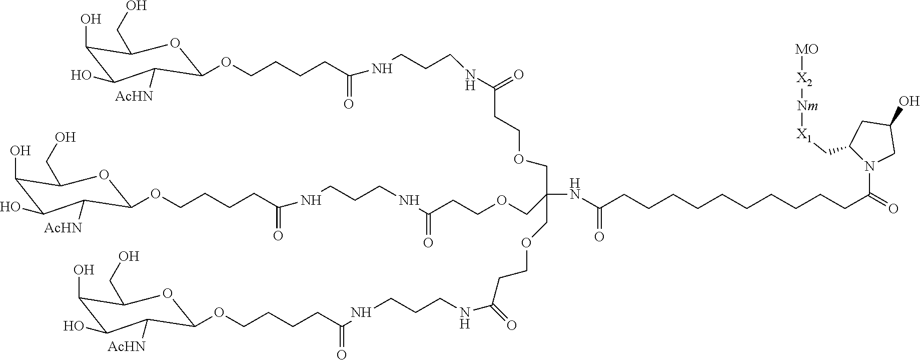

wherein each N is, independently, a modified or unmodified nucleoside and m is from 1 to 5; X₁ and X₂ are each, independently, a phosphodiester linkage or a phosphorothioate linkage; and MO is the modified oligonucleotide.
Embodiment 55. The method of one of embodiments 42 to 50, wherein the compound has the structure:

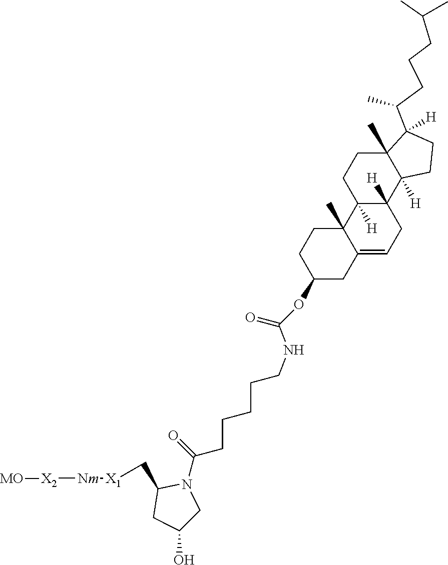

wherein each N is, independently, a modified or unmodified nucleoside and m is from 1 to 5; X₁ and X₂ are each, independently, a phosphodiester linkage or a phosphorothioate linkage; and MO is the modified oligonucleotide.
Embodiment 56. The method of embodiment 54 or 55, wherein at least one of X₁ and X₂ is a phosphodiester linkage.
Embodiment 57. The method of embodiment 54 or 55, wherein each of X₁ and X₂ is a phosphodiester linkage.
Embodiment 58. The method of any one of embodiments 42 to 57, wherein m is 1.
Embodiment 59. The method of any one of embodiments 42 to 57, wherein m is 2, 3, 4, or 5.
Embodiment 60. The method of any one of embodiments 42 to 59, wherein Nₘ is N′ₚN“, wherein each N′ is, independently, an unmodified nucleoside and p is from 0 to 4; and N” is a nucleoside comprising an unmodified sugar moiety.
Embodiment 61. The method of embodiment 60, wherein p is 0.
Embodiment 62. The compound of embodiment 60, wherein p is 1, 2, 3, or 4.
Embodiment 63. The method of any one of embodiments 60 to 62, wherein the unmodified sugar moiety is a β-D-ribose or a β-D-deoxyribose.
Embodiment 64. The method of embodiment 63, wherein the β-D-deoxyribose is β-D-deoxyriboadenosine.
Embodiment 65. The method of any one of embodiments 1 to 64, wherein the compound is present in a pharmaceutical composition.
Embodiment 66 The method of embodiment 65, wherein the pharmaceutical composition comprises a pharmaceutically acceptable diluent.
Embodiment 67. The method of embodiment 66, wherein the pharmaceutically acceptable diluent is an aqueous solution.
Embodiment 68. The method of embodiment 67, wherein the aqueous solution is a saline solution.
Embodiment 69. The method of embodiment 67 or 68, wherein the aqueous solution comprises sodium bicarbonate.

### BRIEF DESCRIPTION OF THE DRAWING

FIG. 1 shows mean ALDOA de-repression following oral (top panel) or subcutaneous (bottom panel) administration of various compounds.

### DETAILED DESCRIPTION

Unless defined otherwise, all technical and scientific terms used herein have the same meaning as is commonly understood by one of skill in the arts to which the invention belongs. Unless specific definitions are provided, the nomenclature utilized in connection with, and the procedures and techniques of, analytical chemistry, synthetic organic chemistry, and medicinal and pharmaceutical chemistry described herein are those well known and commonly used in the art. In the event that there is a plurality of definitions for terms herein, those in this section prevail. Standard techniques may be used for chemical synthesis, chemical analysis, pharmaceutical preparation, formulation and delivery, and treatment of subjects. Certain such techniques and procedures may be found for example in “Carbohydrate Modifications in Antisense Research” Edited by Sangvi and Cook, American Chemical Society, Washington D.C., 1994; and “Remington's Pharmaceutical Sciences,” Mack Publishing Co., Easton, Pa., 18th edition, 1990; and which is hereby incorporated by reference for any purpose. Where permitted, all patents, patent applications, published applications and publications, GENBANK sequences, websites and other published materials referred to throughout the entire disclosure herein, unless noted otherwise, are incorporated by reference in their entirety. Where reference is made to a URL or other such identifier or address, it is understood that such identifiers can change and particular information on the internet can change, but equivalent information can be found by searching the internet. Reference thereto evidences the availability and public dissemination of such information.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

### Definitions

“Subject” means a human selected for treatment or therapy.

“Subject in need thereof” means a subject that is identified as in need of a particular therapy or treatment.

“Subject suspected of having” means a subject exhibiting one or more clinical indicators of a disease.

“Administering” means providing a pharmaceutical agent or composition to a subject, and includes, but is not limited to, administering by a medical professional and self-administering.

“Oral administration” means administration through the mouth of a subject.

“Parenteral administration” means administration through injection or infusion. Parenteral administration includes, but is not limited to, subcutaneous administration, intravenous administration, and intramuscular administration.

“Subcutaneous administration” means administration just below the skin.

“Intravenous administration” means administration into a vein.

“Administered concomitantly” refers to the co-administration of two or more agents to a subject in any manner in which the pharmacological effects of each agent are present in a subject. Concomitant administration does not require that both agents be administered in a single pharmaceutical composition, in the same dosage form, or by the same route of administration. The effects of both agents need not be present at the same time. The effects need only be overlapping for a period of time and need not be coextensive.

“Duration” means the period of time during which an activity or event continues. In certain embodiments, the duration of treatment is the period of time during which doses of a pharmaceutical agent or pharmaceutical composition are administered.

“Therapy” means a disease treatment method. In certain embodiments, therapy includes, but is not limited to, chemotherapy, radiation therapy, or administration of a pharmaceutical agent.

“Treatment” means the application of one or more specific procedures used for the cure or amelioration of a disease. In certain embodiments, the specific procedure is the administration of one or more pharmaceutical agents.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“At risk for developing” means the state in which a subject is predisposed to developing a condition or disease. In certain embodiments, a subject at risk for developing a condition or disease exhibits one or more symptoms of the condition or disease but does not exhibit a sufficient number of symptoms to be diagnosed with the condition or disease. In certain embodiments, a subject at risk for developing a condition or disease exhibits one or more symptoms of the condition or disease, but to a lesser extent required to be diagnosed with the condition or disease.

“Prevent the onset of” means to prevent the development of a condition or disease in a subject who is at risk for developing the disease or condition. In certain embodiments, a subject at risk for developing the disease or condition receives treatment similar to the treatment received by a subject who already has the disease or condition.

“Delay the onset of” means to delay the development of a condition or disease in a subject who is at risk for developing the disease or condition. In certain embodiments, a subject at risk for developing the disease or condition receives treatment similar to the treatment received by a subject who already has the disease or condition.

“Therapeutic agent” means a pharmaceutical agent used for the cure, amelioration or prevention of a disease.

“Dose” means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In certain embodiments, a dose is administered as a slow infusion.

“Dosage unit” means a form in which a pharmaceutical agent is provided. In certain embodiments, a dosage unit is a vial containing lyophilized oligonucleotide. In certain embodiments, a dosage unit is a vial containing reconstituted oligonucleotide.

“Therapeutically effective amount” refers to an amount of a pharmaceutical agent that provides a therapeutic benefit to an animal.

“Pharmaceutical composition” means a mixture of substances suitable for administering to an individual that includes a pharmaceutical agent. For example, a pharmaceutical composition may comprise a sterile aqueous solution.

“Pharmaceutical agent” means a substance that provides a therapeutic effect when administered to a subject.

“Active pharmaceutical ingredient” means the substance in a pharmaceutical composition that provides a desired effect.

“Pharmaceutically acceptable salt” means a physiologically and pharmaceutically acceptable salt of a compound provided herein, i.e., a salt that retains the desired biological activity of the compound and does not have undesired toxicological effects when administered to a subject. Nonlimiting exemplary pharmaceutically acceptable salts of compounds provided herein include sodium and potassium salt forms. The terms “compound,” “oligonucleotide,” and “modified oligonucleotide” as used herein include pharmaceutically acceptable salts thereof unless specifically indicated otherwise.

“Saline solution” means a solution of sodium chloride in water.

“Improved organ function” means a change in organ function toward normal limits. In certain embodiments, organ function is assessed by measuring molecules found in a subject's blood or urine. For example, in certain embodiments, improved liver function is measured by a reduction in blood liver transaminase levels. In certain embodiments, improved kidney function is measured by a reduction in blood urea nitrogen, a reduction in proteinuria, a reduction in albuminuria, etc.

“Acceptable safety profile” means a pattern of side effects that is within clinically acceptable limits.

“Side effect” means a physiological response attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. Such side effects may be detected directly or indirectly. For example, increased aminotransferase levels in serum may indicate liver toxicity or liver function abnormality. For example, increased bilirubin may indicate liver toxicity or liver function abnormality.

“Injection site reaction” means inflammation or abnormal redness of skin at a site of injection in an individual.

“Subject compliance” means adherence to a recommended or prescribed therapy by a subject.

“Comply” means the adherence with a recommended therapy by a subject.

“Recommended therapy” means a treatment recommended by a medical professional to treat, ameliorate, delay, or prevent a disease.

“Oligonucleotide” means a compound comprising a plurality of linked nucleosides, each of which can be modified or unmodified, independent from one another.

“Modified oligonucleotide” means a single-stranded oligonucleotide having one or more modifications relative to a naturally occurring terminus, sugar, nucleobase, and/or internucleoside linkage. A modified oligonucleotide may comprise unmodified nucleosides.

“Anti-miR” means a modified oligonucleotide having a nucleobase sequence complementary to a microRNA.

“Targeting” means the process of design and selection of nucleobase sequence that will hybridize to a target nucleic acid.

“Targeted to” means having a nucleobase sequence that will allow hybridization to a target nucleic acid.

“Modulation” means a perturbation of function, amount, or activity. In certain embodiments, modulation means an increase in function, amount, or activity. In certain embodiments, modulation means a decrease in function, amount, or activity.

“Expression” means any functions and steps by which a gene's coded information is converted into structures present and operating in a cell.

“Nucleobase sequence” means the order of contiguous nucleobases in an oligomeric compound or nucleic acid, typically listed in a 5′ to 3′ orientation, independent of any sugar, linkage, and/or nucleobase modification.

“Contiguous nucleobases” means nucleobases immediately adjacent to each other in a nucleic acid.

“Nucleobase complementarity” means the ability of two nucleobases to pair non-covalently via hydrogen bonding.

“Complementary” means that one nucleic acid is capable of hybridizing to another nucleic acid or oligonucleotide. In certain embodiments, complementary refers to an oligonucleotide capable of hybridizing to a target nucleic acid.

“Fully complementary” means each nucleobase of an oligonucleotide is capable of pairing with a nucleobase at each corresponding position in a target nucleic acid. In certain embodiments, an oligonucleotide is fully complementary (also referred to as 100% complementary) to a microRNA, i.e. each nucleobase of the oligonucleotide is complementary to a nucleobase at a corresponding position in the microRNA. A modified oligonucleotide may be fully complementary to a microRNA and have a number of linked nucleosides that is less than the length of the microRNA. For example, an oligonucleotide with 10 linked nucleosides, where each nucleobase of the oligonucleotide is complementary to a nucleobase at a corresponding position in a microRNA, is fully complementary to the microRNA.

“Percent complementarity” means the percentage of nucleobases of an oligonucleotide that are complementary to an equal-length portion of a target nucleic acid. Percent complementarity is calculated by dividing the number of nucleobases of the oligonucleotide that are complementary to nucleobases at corresponding positions in the target nucleic acid by the total number of nucleobases in the oligonucleotide.

“Percent identity” means the number of nucleobases in a first nucleic acid that are identical to nucleobases at corresponding positions in a second nucleic acid, divided by the total number of nucleobases in the first nucleic acid. In certain embodiments, the first nucleic acid is a microRNA and the second nucleic acid is a microRNA. In certain embodiments, the first nucleic acid is an oligonucleotide and the second nucleic acid is an oligonucleotide.

“Hybridize” means the annealing of complementary nucleic acids that occurs through nucleobase complementarity.

“Mismatch” means a nucleobase of a first nucleic acid that is not capable of Watson-Crick pairing with a nucleobase at a corresponding position of a second nucleic acid.

“Identical” in the context of nucleobase sequences, means having the same nucleobase sequence, independent of sugar, linkage, and/or nucleobase modifications and independent of the methyl state of any pyrimidines present.

“MicroRNA” means an endogenous non-coding RNA between 18 and 25 nucleobases in length, which is the product of cleavage of a pre-microRNA by the enzyme Dicer. Examples of mature microRNAs are found in the microRNA database known as miRBase (http://microrna.sanger.ac.uk/). In certain embodiments, microRNA is abbreviated as “microRNA” or “miR.”

“microRNA-regulated transcript” means a transcript that is regulated by a microRNA.

“Seed sequence” means a nucleobase sequence comprising nucleobases 2 to 7 of the 5′-end of a mature microRNA sequence.

“Seed match sequence” means a nucleobase sequence that is complementary to a seed sequence and is the same length as the seed sequence.

“Naturally occurring internucleoside linkage” means a 3′ to 5′ phosphodiester linkage between nucleosides.

“Natural sugar” means a sugar found in DNA (2′-H) or RNA (2′-OH).

“Internucleoside linkage” means a covalent linkage between adjacent nucleosides.

“Linked nucleosides” means nucleosides joined by a covalent linkage.

“Nucleobase” means a heterocyclic moiety capable of non-covalently pairing with another nucleobase.

“Nucleoside” means a nucleobase linked to a sugar moiety.

“Nucleotide” means a nucleoside having a phosphate group covalently linked to the sugar portion of a nucleoside.

“Compound comprising a modified oligonucleotide consisting of” a number of linked nucleosides means a compound that includes a modified oligonucleotide having the specified number of linked nucleosides. Thus, the compound may include additional substituents or conjugates. Unless otherwise indicated, the compound does not include any additional nucleosides beyond those of the modified oligonucleotide.

“Modified nucleoside” means a nucleoside having any change from a naturally occurring nucleoside. A modified nucleoside may have a modified sugar, and an unmodified nucleobase. A modified nucleoside may have a modified sugar and a modified nucleobase. A modified nucleoside may have a natural sugar and a modified nucleobase. In certain embodiments, a modified nucleoside is a bicyclic nucleoside. In certain embodiments, a modified nucleoside is a non-bicyclic nucleoside.

“2′-modified nucleoside” means a nucleoside comprising a sugar with any modification at the position equivalent to the 2′ position of the furanosyl ring as the positions are numbered in 2-deoxyribose or ribose. It is to be understood that 2′-modified nucleosides include, without limitation, nucleosides comprising bicyclic sugar moieties.

“Modified internucleoside linkage” means any change from a naturally occurring internucleoside linkage.

“Phosphorothioate internucleoside linkage” means a linkage between nucleosides where one of the non-bridging atoms is a sulfur atom, i.e. OP(O)(S)O—. For the avoidance of doubt, the sulfur atom may be protonated or associated with a counterion, such as Na⁺, K⁺, etc.

“Phosphodiester linkage” means a linkage between nucleosides having the structure —OP(O)₂O—. For the avoidance of doubt, one of the non-bridging oxygens may be protonated or associated with a counterion, such as Na⁺, K⁺, etc.

“Unmodified nucleobase” means the naturally occurring heterocyclic bases of RNA or DNA: the purine bases adenine (A) and guanine (G), and the pyrimidine bases thymine (T), cytosine (C) (including 5-methylcytosine), and uracil (U).

“5-methylcytosine” means a cytosine comprising a methyl group attached to the 5 position of the cytosine ring.

“Non-methylated cytosine” means a cytosine that does not have a methyl group attached to the 5 position of the cytosine ring.

“Modified nucleobase” means any nucleobase that is not an unmodified nucleobase.

“Sugar moiety” means a naturally occurring furanosyl or a modified sugar moiety.

“Modified sugar moiety” means a substituted sugar moiety or a sugar surrogate.

“2′-O-methyl sugar” or “2′-OMe sugar” means a sugar having a O-methyl modification at the 2′ position.

“2′-O-methoxyethyl sugar” or “2′-MOE sugar” means a sugar having a 0-methoxyethyl modification at the 2′ position.

“2′-O-fluoro” or “2′-F” means a sugar having a fluoro modification of the 2′ position.

“Bicyclic sugar moiety” means a modified sugar moiety comprising a 4 to 7 membered ring (including by not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl. Nonlimiting exemplary bicyclic sugar moieties include LNA, ENA, cEt, S-cEt, and R-cEt.

“Locked nucleic acid (LNA) sugar moiety” means a substituted sugar moiety comprising a (CH₂)—O bridge between the 4′ and 2′ furanose ring atoms.

“ENA sugar moiety” means a substituted sugar moiety comprising a (CH₂)₂—O bridge between the 4′ and 2′ furanose ring atoms.

“Constrained ethyl (cEt) sugar moiety” means a substituted sugar moiety comprising a CH(CH₃)—O bridge between the 4′ and the 2′ furanose ring atoms. In certain embodiments, the CH(CH₃)—O bridge is constrained in the S orientation. In certain embodiments, the CH(CH₃)—O bridge is constrained in the R orientation.

“S-cEt sugar moiety” means a substituted sugar moiety comprising an S-constrained CH(CH₃)—O bridge between the 4′ and the 2′ furanose ring atoms.

“R-cEt sugar moiety” means a substituted sugar moiety comprising an R-constrained CH(CH₃)—O bridge between the 4′ and the 2′ furanose ring atoms.

“2′-O-methyl nucleoside” means a modified nucleoside having a 2′-O-methyl sugar modification.

“2′-O-methoxyethyl nucleoside” means a modified nucleoside having a 2′-O-methoxyethyl sugar modification. A 2′-O-methoxyethyl nucleoside may comprise a modified or unmodified nucleobase.

“2′-fluoro nucleoside” means a modified nucleoside having a 2′-fluoro sugar modification. A 2′-fluoro nucleoside may comprise a modified or unmodified nucleobase.

“Bicyclic nucleoside” means a modified nucleoside having a bicyclic sugar moiety. A bicyclic nucleoside may have a modified or unmodified nucleobase.

“cEt nucleoside” means a nucleoside comprising a cEt sugar moiety. A cEt nucleoside may comprise a modified or unmodified nucleobase.

“S-cEt nucleoside” means a nucleoside comprising an S-cEt sugar moiety.

“R-cEt nucleoside” means a nucleoside comprising an R-cEt sugar moiety.

“β-D-deoxyribonucleoside” means a naturally occurring DNA nucleoside.

“β-D-ribonucleoside” means a naturally occurring RNA nucleoside.

“LNA nucleoside” means a nucleoside comprising a LNA sugar moiety.

“ENA nucleoside” means a nucleoside comprising an ENA sugar moiety.

A “linking group” as used herein refers to an atom or group of atoms that attach a first chemical entity to a second chemical entity via one or more covalent bonds.

A “linker” as used herein, refers to an atom or group of atoms that attach one or more ligands to a modified or unmodified nucleoside via one or more covalent bonds. The modified or unmodified nucleoside may be part of a modified oligonucleotide as described herein, or may be attached to a modified oligonucleotide through a phosphodiester or phosphorothioate bond. In some embodiments, the linker attaches one or more ligands to the 3′ end of a modified oligonucleotide. In some embodiments, the linker attaches one or more ligands to the 5′ end of a modified oligonucleotide. In some embodiments, the linker attaches one or more ligands to a modified or unmodified nucleoside that is attached to the 3′ end of a modified oligonucleotide. In some embodiments, the linker attaches one or more ligands to a modified or unmodified nucleoside that is attached to the 5′ end of a modified oligonucleotide. When the linker attaches one or more ligands to the 3′ end of a modified oligonucleotide or to a modified or unmodified nucleoside attached to the 3′ end of a modified oligonucleotide, in some embodiments, the attachment point for the linker may be the 3′ carbon of a modified or unmodified sugar moiety. When the linker attaches one or more ligands to the 5′ end of a modified oligonucleotide or to a modified or unmodified nucleoside attached to the 5′ end of a modified oligonucleotide, in some embodiments, the attachment point for the linker may be the 5′ carbon of a modified or unmodified sugar moiety.

### Overview

Oral administration of oligonucleotides has several features that may be advantageous to subcutaneous administration, for example, improved patient compliance, improved convenience of dosing, and lack of subcutaneous injection site reactions.

To date, oral administration of oligonucleotides, including anti-miR compounds, has not been well-characterized. Accordingly, oral administration of modified oligonucleotides was evaluated in experimental animal models. The modified oligonucleotides varied in nucleobase sequence, length, chemical modification patterns, and presence of conjugate moieties. Unexpectedly, it was observed that oral administration of certain modified oligonucleotides exhibited pharmacodynamic activity comparable to that observed following subcutaneous administration. Moreover, robust pharmacodynamic activity was observed where the amount of modified oligonucleotide detected in the target tissue relatively low compared to that detected for the same compound administered subcutaneously.

Accordingly, provided herein are methods of oral administration to a subject of a compound comprising a modified oligonucleotide complementary to a target RNA. The target RNA may be, for example, a microRNA, a pre-messenger RNA, a messenger RNA, or a long non-coding RNA.

### Certain Methods

Provided herein are methods of inhibiting the activity of a target RNA, comprising administering to a subject a compound comprising a modified oligonucleotide complementary to the target RNA, or a pharmaceutically acceptable salt thereof, wherein the modified oligonucleotide has a length of 6 to 25 linked nucleotides and wherein the administration is oral administration. In certain embodiments, the target RNA is a microRNA. In certain embodiments, the target RNA is a pre-messenger RNA. In certain embodiments, the target RNA is a messenger RNA. In certain embodiments, the target RNA is a long non-coding RNA.

In certain embodiments, the target RNA is present in the kidney and the compound consists of the modified oligonucleotide. In certain embodiments, the target RNA is present in the liver and the compound comprises the modified oligonucleotide linked to a conjugate moiety.

In certain embodiments, the subject has a disease mediated by the target RNA. In certain embodiments, the oral administration of the compound improves one or more symptoms of the disease mediated by the target RNA.

Provided herein are methods of inhibiting the activity of a microRNA, comprising administering to a subject a compound comprising a modified oligonucleotide complementary to the microRNA, or a pharmaceutically acceptable salt thereof, wherein the modified oligonucleotide has a length of 6 to 25 linked nucleotides and wherein the administration is oral administration. In certain embodiments, the microRNA is present in the kidney and the compound consists of the modified oligonucleotide. In certain embodiments, the microRNA is present in the liver and the compound comprises the modified oligonucleotide linked to a conjugate moiety.

In certain embodiments, the subject has a disease mediated by the microRNA. In certain embodiments, the oral administration of the compound improves one or more symptoms of the disease mediated by the microRNA.

In certain embodiments, the methods provided herein comprise contacting a hepatocyte with a compound provided herein. In certain embodiments, the methods provided herein comprise contacting a kidney cell with a compound provided herein.

Provided herein are methods for oral administration of compounds comprising a modified oligonucleotide complementary to a target RNA, for use in therapy.

Provided herein are methods for oral administration of compounds comprising a modified oligonucleotide complementary to a microRNA, for use in therapy.

### Certain Compounds and Compositions

In certain embodiments, a method provided herein comprises oral administration of a compound comprising a modified oligonucleotide. In certain embodiments, a method provided herein comprises oral administration of a compound consisting of a modified oligonucleotide.

In certain embodiments, a modified oligonucleotide has a length of 6 to 25 linked nucleosides. In certain embodiments, a modified oligonucleotide has a length of 6 to 21 linked nucleosides. In certain embodiments, a modified oligonucleotide has a length of 6 to 18 linked nucleosides. In certain embodiments, a modified oligonucleotide has a length of 6 to 15 linked nucleosides. In certain embodiments, a modified oligonucleotide has a length of 6 to 12 linked nucleosides. In certain embodiments, a modified oligonucleotide has a length of 8 to 10 linked nucleosides. In certain embodiments, a modified oligonucleotide has a length of 8 to 12 linked nucleosides. In certain embodiments, a modified oligonucleotide has a length of 8 to 13 linked nucleosides. In certain embodiments, a modified oligonucleotide is 6 linked nucleosides in length. In certain embodiments, a modified oligonucleotide is 7 linked nucleosides in length. In certain embodiments, a modified oligonucleotide is 8 linked nucleosides in length. In certain embodiments, a modified oligonucleotide is 9 linked nucleosides in length. In certain embodiments, a modified oligonucleotide is 10 linked nucleosides in length. In certain embodiments, a modified oligonucleotide is 11 linked nucleosides in length. In certain embodiments, a modified oligonucleotide is 12 linked nucleosides in length. In certain embodiments, a modified oligonucleotide is 13 linked nucleosides in length. In certain embodiments, a modified oligonucleotide is 14 linked nucleosides in length. In certain embodiments, a modified oligonucleotide is 15 linked nucleosides in length. In certain embodiments, a modified oligonucleotide is 16 linked nucleosides in length. In certain embodiments, a modified oligonucleotide is 17 linked nucleosides in length. In certain embodiments, a modified oligonucleotide is 18 linked nucleosides in length. In certain embodiments, a modified oligonucleotide is 19 linked nucleosides in length. In certain embodiments, a modified oligonucleotide is 20 linked nucleosides in length. In certain embodiments, a modified oligonucleotide is 21 linked nucleosides in length. In certain embodiments, a modified oligonucleotide is 22 linked nucleosides in length. In certain embodiments, a modified oligonucleotide is 23 linked nucleosides in length. In certain embodiments, a modified oligonucleotide is 24 linked nucleosides in length. In certain embodiments, a modified oligonucleotide is 25 linked nucleosides in length.

In certain embodiments, the nucleobase sequence of a modified oligonucleotide is at least 80%, at least 85%, at least 90%, at least 91%, at least 92%, at least 95%, at least 96%, or 100% complementary to the nucleobase sequence of a target RNA. In certain embodiments, a modified oligonucleotide is at least 90%, at least 93%, at least 94%, at least 95%, or 100% complementary to a target RNA.

In certain embodiments, a modified oligonucleotide is at least 90%, at least 93%, at least 94%, at least 95%, or 100% complementary to the nucleobase sequence of a microRNA.

In each internucleoside linkage of a modified oligonucleotide (e.g., each phosphorothioate linkage and each phosphodiester linkage), a non-bridging heteroatom (e.g., an S⁻ or O⁻) may be protonated or associated with a counterion such as etc. A pharmaceutically acceptable salt of a compound may comprise fewer cationic counterions (such as Na⁺, K⁺, etc) than there are phosphorothioate and/or phosphodiester linkages per molecule (i.e., some phosphorothioate and/or phosphodiester linkages are protonated and some are associated with counterions). For example, a pharmaceutically acceptable salt of modified oligonucleotide that is 9 linked nucleotides in length may comprise fewer than 8 cationic counterions (such as Na⁺, K⁺, etc.) per molecule of modified oligonucleotide. That is, in some embodiments, the pharmaceutically acceptable salt of may comprise, on average, 1, 2, 3, 4, 5, 6, or 7 cationic counterions per molecule of modified oligonucleotide, with the remaining phosphorothioate and/or phosphodiester linkages being protonated.

Provided herein are pharmaceutical compositions comprising a compound provided herein, and a pharmaceutically acceptable diluent. In certain embodiments, the pharmaceutically acceptable diluent is an aqueous solution. In certain embodiments, the aqueous solution is a saline solution. As used herein, pharmaceutically acceptable diluents are understood to be sterile diluents.

### Certain Modifications

A modified oligonucleotide may comprise one or more modifications to a nucleobase, sugar, and/or internucleoside linkage. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases.

In certain embodiments, a modified oligonucleotide comprises one or more modified nucleosides. In certain embodiments, a modified nucleoside is a stabilizing nucleoside. An example of a stabilizing nucleoside is a 2′-modified nucleoside.

In certain embodiments, a modified nucleoside comprises a modified sugar moiety. In certain embodiments, a modified nucleoside comprising a modified sugar moiety comprises an unmodified nucleobase. In certain embodiments, a modified sugar comprises a modified nucleobase. In certain embodiments, a modified nucleoside is a 2′-modified nucleoside.

In certain embodiments, a 2′-modified nucleoside comprises a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In certain such embodiments, the bicyclic sugar moiety is an L sugar in the beta configuration.

In certain embodiments, the bicyclic sugar moiety comprises a bridge group between the 2′ and the 4′-carbon atoms. Nucleosides comprising bicyclic sugar moieties are referred to as bicyclic nucleosides or BNAs. In certain embodiments, bicyclic nucleosides include, but are not limited to, (A) α-L-Methyleneoxy (4′-CH₂—O-2′) BNA; (B) β-D-Methyleneoxy (4′-CH₂—O-2′) BNA; (C) Ethyleneoxy (4′-(CH₂)₂-O-2′) BNA; (D) Aminooxy (4′-CH₂—O—N(R)-2′) BNA; (E) Oxyamino (4′-CH₂—N(R)—O-2′) BNA; (F) Methyl(methyleneoxy) (4′-CH(CH₃)—O-2′) BNA (also referred to as constrained ethyl or cEt); (G) methylene-thio (4′-CH₂—S-2′) BNA; (H) methylene-amino (4′-CH₂—N(R)-2′) BNA; (I) methyl carbocyclic (4′-CH₂—CH(CH₃)-2′) BNA; (J) c-MOE (4′-CH₂—OMe-2′) BNA and (K) propylene carbocyclic (4′-(CH₂)₃-2′) BNA as depicted below.

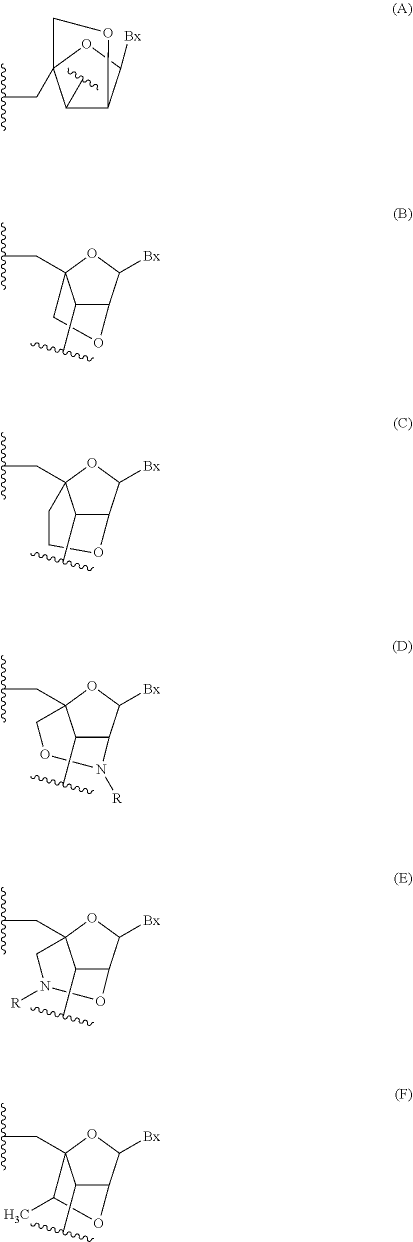

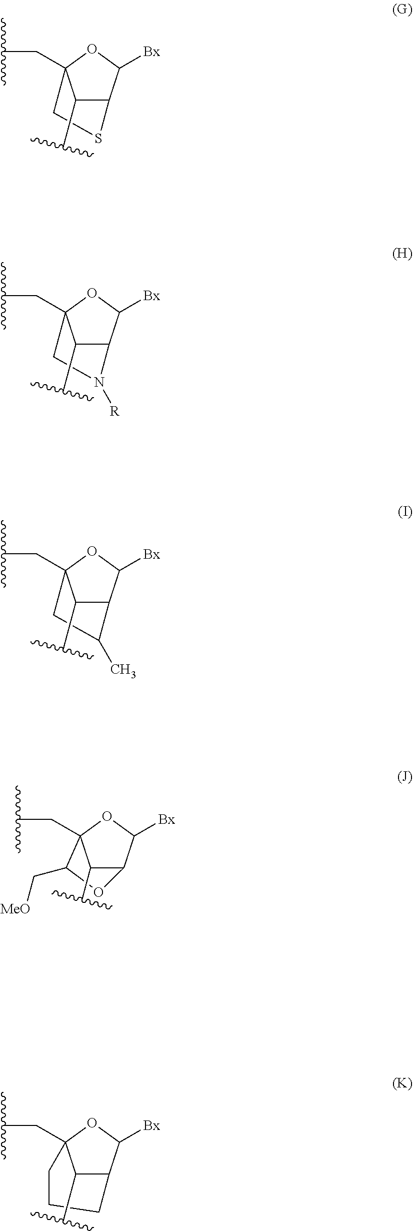

wherein Bx is a nucleobase moiety and R is, independently, H, a protecting group, or C₁-C₁₂ alkyl.

In certain embodiments, a 2′-modified nucleoside comprises a 2′-substituent group selected from F, OCF₃, O—CH₃, OCH₂CH₂OCH₃, 2′—O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In certain embodiments, a 2′-modified nucleoside comprises a 2′-substituent group selected from F, O—CH₃, and OCH₂CH₂OCH₃.

In certain embodiments, a modified oligonucleotide comprises one or more internucleoside modifications. In certain such embodiments, each internucleoside linkage of a modified oligonucleotide is a modified internucleoside linkage. In certain embodiments, a modified internucleoside linkage comprises a phosphorus atom.

In certain embodiments, a modified oligonucleotide comprises at least one phosphorothioate internucleoside linkage. In certain embodiments, each internucleoside linkage of a modified oligonucleotide is a phosphorothioate internucleoside linkage.

In certain embodiments, a modified oligonucleotide comprises one or more modified nucleobases. In certain embodiments, a modified nucleobase is selected from 7-deazaguanine, 7-deazaadenine, hypoxanthine, xanthine, 7-methylguanine, 2-aminopyridine and 2-pyridone. In certain embodiments, a modified nucleobase is selected from 5-substituted pyrimidines, 6-azapyrimidines and N-2, N-6 and 0-6 substituted purines, including 2 aminopropyladenine, 5-propynyluracil and 5-propynylcytosine.

### Certain Conjugate Structures

In certain embodiments, provided herein are compounds comprising a modified oligonucleotide and a conjugate moiety, wherein the conjugate moiety improves the delivery of the compound to a target cell type.

Conjugated compounds may be described by Structure C:

Lₙ-linker-X₁—Nₘ-X₂-MO;

wherein each L is, independently, a ligand and n is from 1 to 10; each N is, independently, a modified or unmodified nucleoside and m is from 1 to 5; X₁ is a phosphodiester linkage or a phosphorothioate linkage; X₂ is a phosphodiester linkage or a phosphorothioate linkage; and MO is a modified oligonucleotide.

For example, the anti-miR-122 compound RG6650 may be described by the following embodiment of Structure C:

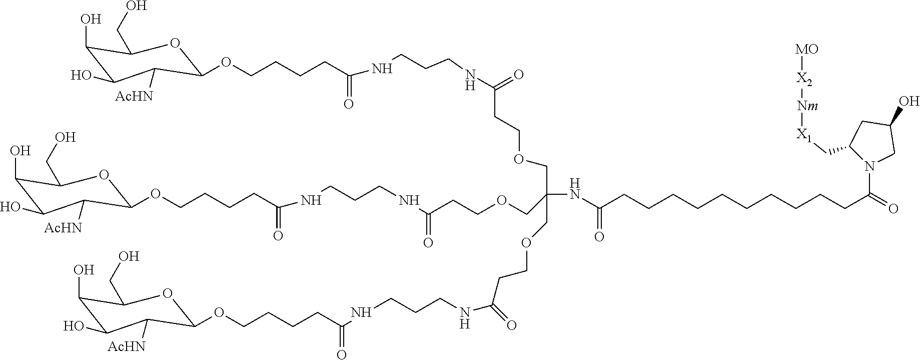

wherein the MO is a modified oligonucleotide and has the structure U_{S}C_{S}AC_{S}AC_{S}TC_{S}C_{S} (RG4773), where nucleosides not followed by a subscript are β-D-deoxyribonucleosides, nucleosides followed by a subscript “S” are S-cEt nucleosides, and each internucleoside linkage is a phosphorothioate internucleoside linkage; wherein X₁ is a phosphodiester linkage; m is 1; N is a β-D-deoxyriboadenosine; X₂ is a phosphodiester linkage; and wherein the conjugate moiety is linked to the 3′ terminus of the modified oligonucleotide.

In certain embodiments, one or more ligands of Structure C may be a ligand that, like the GalNAc moiety, facilitates uptake in the liver. Such ligands include cholesterol, and other ligands having affinity for the asialoglycoprotein receptor (ASGPR), including but not limited to galactose or a galactose derivative. In certain embodiments, a ligand having affinity for the ASGPR is N-acetylgalactosamine, galactose, galactosamine, N-formylgalactosamine, N-propionyl-galactosamine, N-n-butanoylgalactosamine, or N-iso-butanoyl-galactosamine.

In certain embodiments, when n is greater than 1, the linker comprises a scaffold capable of linking more than one L to the remainder of the compound (i.e., to the modified oligonucleotide (MO), to X₁—Nₘ—X₂-MO, to X—Nₘ—Y-MO, etc.). In some such embodiments, the Lₙ-linker portion of the compound (such as a compound of Structure A, B, C, or D) comprises Structure E:

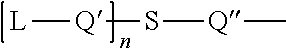

wherein each L is, independently, a ligand; n is from 1 to 10; S is a scaffold; and Q′ and Q″ are, independently, linking groups.

In certain embodiments, each Q′ and Q″ is independently selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid.

In certain embodiments, a scaffold links 2, 3, 4, or 5 ligands to a modified oligonucleotide. In certain embodiments, a scaffold links 3 ligands to a modified oligonucleotide.

A nonlimiting exemplary Structure E is Structure E(i):

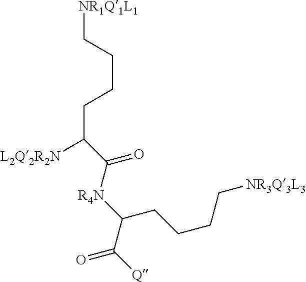

wherein L₁, L₂, and L₃ are each, independently, a ligand; Q′₁, Q′₂, Q′₃, and Q″ are each, independently, a linking group; and R₁, R2, R3, and R4 are each, independently, selected from H, C₁-C₆ alkyl, and substituted C₁-C₆ alkyl.

In some embodiments, Q′₁, Q′₂, Q′₃, and Q″ are each, independently, selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid. In some embodiments, R₁, R₂, R₃, and R₄ are each, independently, selected from H, methyl, ethyl, propyl, isopropyl, and butyl. In some embodiments, R₁, R₂, R₃, and R₄ are each selected from H and methyl.

A further nonlimiting exemplary Structure E is Structure E(ii):

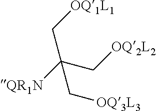

wherein L₁, L₂, and L₃ are each, independently, a ligand; Q′₁, Q′₂, Q′₃, and Q″ are each, independently, a linking group; and R₁ is selected from H, C₁-C₆ alkyl, and substituted C₁-C₆ alkyl.

In some embodiments, Q′₁, Q′₂, Q′₃, and Q″ are each, independently, selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid. In some embodiments, R₁ is selected from H, methyl, ethyl, propyl, isopropyl, and butyl. In some embodiments, R₁ is H or methyl.

A further nonlimiting exemplary Structure E is Structure E(iii):

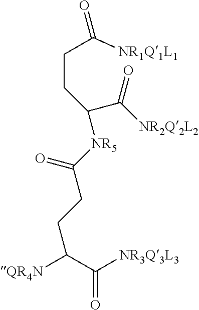

wherein L₁, L₂, and L₃ are each, independently, a ligand; Q′₁, Q′₂, Q′₃, and Q″ are each, independently, a linking group; and R₁, R₂, R₃, R₄, and R₅ are each, independently, selected from H, C₁-C₆ alkyl, and substituted C₁-C₆ alkyl.

In some embodiments, Q′₁, Q′₂, Q′₃, and Q″ are each, independently, selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid. In some embodiments, R₁, R₂, R₃, R₄, and R₅ are each, independently, selected from H, methyl, ethyl, propyl, isopropyl, and butyl. In some embodiments R₁, R₂, R₃, R₄, and R₅ are each selected from H and methyl.

A further nonlimiting exemplary Structure E is Structure E(iv):

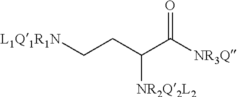

wherein L₁ and L₂ are each, independently, a ligand; Q′₁, Q′₂, and Q″ are each, independently, a linking group; and R₁, R₂, and R₃ are each, independently, selected from H, C₁-C₆ alkyl, and substituted C₁-C₆ alkyl.

In some embodiments, Q′₁, Q′₂, and Q″ are each, independently, selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid. In some embodiments, R₁, R₂, and R₃ are each, independently, selected from H, methyl, ethyl, propyl, isopropyl, and butyl. In some embodiments R₁, R₂, and R₃ are each selected from H and methyl.

A further nonlimiting exemplary Structure E is Structure E(v):

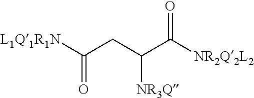

wherein L₁ and L₂ are each, independently, a ligand; Q′₁, Q′₂, and Q″ are each, independently, a linking group; and R₁, R₂, and R₃ are each, independently, selected from H, C₁-C₆ alkyl, and substituted C₁-C₆ alkyl.

In some embodiments, Q′₁, Q′₂, and Q″ are each, independently, selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid. In some embodiments, R₁, R₂, and R₃ are each, independently, selected from H, methyl, ethyl, propyl, isopropyl, and butyl. In some embodiments R₁, R₂, and R₃ are each selected from H and methyl.

A further nonlimiting exemplary Structure E is Structure E(vi):

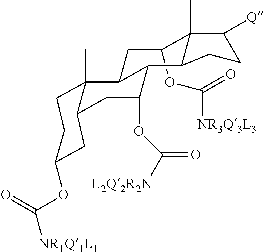

wherein L₁, L₂, and L₃ are each, independently, a ligand; Q′₁, Q′₂, Q′₃, and Q″ are each, independently, a linking group; and R₁, R₂, and R₃ are each, independently, selected from H, C₁-C₆ alkyl, and substituted C₁-C₆ alkyl.

In some embodiments, Q′₁, Q′₂, Q′₃, and Q″ are each, independently, selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid. In some embodiments, R₁, R₂, and R₃ are each, independently, selected from H, methyl, ethyl, propyl, isopropyl, and butyl. In some embodiments R₁, R₂, and R₃ are each selected from H and methyl.

A further nonlimiting exemplary Structure E is Structure E(vii):

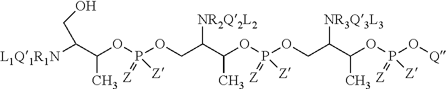

wherein L₁, L₂, and L₃ are each, independently, a ligand; Q′₁, Q′₂, Q′₃, and Q″ are each, independently, a linking group; R₁, R₂, and R₃ are each, independently, selected from H, C₁-C₆ alkyl, and substituted C₁-C₆ alkyl; and Z and Z′ are each independently selected from O and S.

In some embodiments, Q′₁, Q′₂, Q′₃, and Q″ are each, independently, selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid. In some embodiments, R₁, R₂, and R₃ are each, independently, selected from H, methyl, ethyl, propyl, isopropyl, and butyl. In some embodiments R₁, R₂, and R₃ are each selected from H and methyl. In some embodiments, Z or Z′ on at least one P atom is S, and the other Z or Z′ is O (i.e., a phosphorothioate linkage). In some embodiments, each —OP(Z)(Z′)O— is a phosphorothioate linkage. In some embodiments, Z and Z′ are both O on at least one P atom (i.e., a phosphodiester linkage). In some embodiments, each —OP(Z)(Z′)O— is a phosphodiester linkage.

A further nonlimiting exemplary Structure E is Structure E(viii):

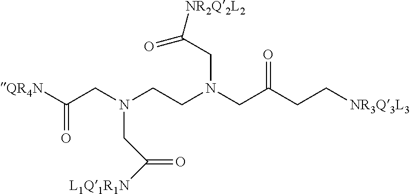

wherein L₁, L₂, and L₃ are each, independently, a ligand; Q′₁, Q′₂, Q′₃, and Q″ are each, independently, a linking group; and R₁, R₂, R₃, and R₄ are each, independently, selected from H, C₁-C₆ alkyl, and substituted C₁-C₆ alkyl.

In some embodiments, Q′₁, Q′₂, Q′₃, and Q″ are each, independently, selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid. In some embodiments, R₁, R₂, R₃, and R₄ are each, independently, selected from H, methyl, ethyl, propyl, isopropyl, and butyl. In some embodiments R₁, R₂, R₃, and R₄ are each selected from H and methyl.

Nonlimiting exemplary scaffolds and/or linkers comprising scaffolds, and synthesis thereof, are described, e.g., PCT Publication No. WO 2013/033230, U.S. Pat. No. 8,106,022 B2, U.S. Publication No. 2012/0157509 A1; U.S. Pat. Nos. 5,994,517; 7,491,805 B2; 8,313,772 B2; Manoharan, M., Chapter 16, Antisense Drug Technology, Crooke, S. T., Marcel Dekker, Inc., 2001, 391-469.

In certain embodiments, the Lₙ-linker portion of the compound comprises Structure F:

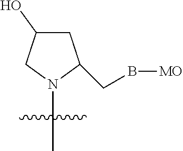

wherein:

B is selected from —O—, —S—, —N(R^{N})—, —Z—P(Z′)(Z″)O—, —Z—P(Z)(Z″)O—Nₘ—X—, and —Z—P(Z)(Z″)O—Nₘ—Y—;

MO is a modified oligonucleotide;

R^{N} is selected from H, methyl, ethyl, propyl, isopropyl, butyl, and benzyl;

Z, Z′, and Z″ are each independently selected from O and S;

each N is, independently, a modified or unmodified nucleoside;

m is from 1 to 5;

X is selected from a phosphodiester linkage and a phosphorothioate linkage;

Y is a phosphodiester linkage; and

the wavy line indicates the connection to the rest of the linker and ligand(s).

In certain embodiments, the wavy line indicates a connection to Structure E, above.

In certain embodiments, n is from 1 to 5, 1 to 4, 1 to 3, or 1 to 2. In certain embodiments, n is 1. In certain embodiments, n is 2. In certain embodiments, n is 3. In certain embodiments, n is 4. In certain embodiments, n is 5.

In certain embodiments, the Lₙ-linker portion of the compound comprises Structure G:

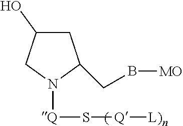

wherein:
B is selected from —O—, —S—, —N(R^{N})—, —Z—P(Z′)(Z″)O—, —Z—P(Z′)(Z″)O—Nₘ—X—, and —Z—P(Z′)(Z″)O—Nₘ—Y—;

MO is a modified oligonucleotide;

R^{N} is selected from H, methyl, ethyl, propyl, isopropyl, butyl, and benzyl;

Z, Z′, and Z″ are each independently selected from O and S;

each N is, independently, a modified or unmodified nucleoside;

m is from 1 to 5;

X is selected from a phosphodiester linkage and a phosphorothioate linkage;

Y is a phosphodiester linkage;

each L is, independently, a ligand; n is from 1 to 10; S is a scaffold; and Q′ and Q″ are, independently, linking groups.

In certain embodiments, each Q′ and Q″ are independently selected from a peptide, an ether, polyethylene glycol, an alkyl, a C₁-C₂₀ alkyl, a substituted C₁-C₂₀ alkyl, a C₂-C₂₀ alkenyl, a substituted C₂-C₂₀ alkenyl, a C₂-C₂₀ alkynyl, a substituted C₂-C₂₀ alkynyl, a C₁-C₂₀ alkoxy, a substituted C₁-C₂₀ alkoxy, amino, amido, a pyrrolidine, 8-amino-3,6-dioxaoctanoic acid (ADO), succinimidyl 4-(N-maleimidomethyl) cyclohexane-1-carboxylate, and 6-aminohexanoic acid.

A nonlimiting exemplary Lₙ-linker portion (e.g., of Structure F or G) of a compound is shown in Structure H below:

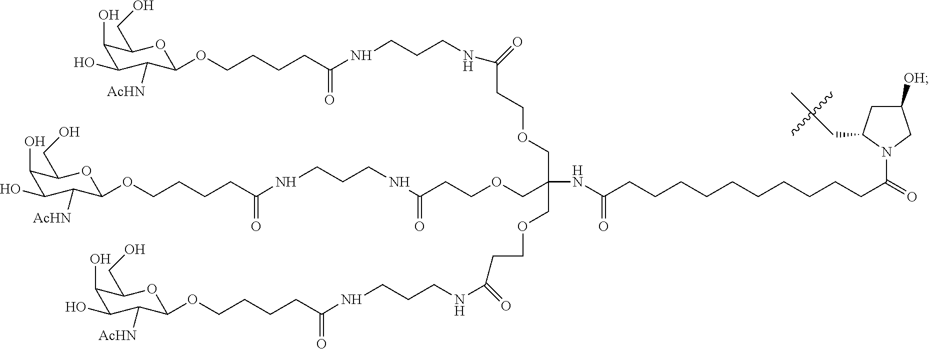 [SMILES: *C[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

wherein the wavy line indicates attachment to the modified oligonucleotide, to X₁, e.g. in Structure B, or to X or Y, e.g., in Structure C, or D.

In certain embodiments, a compound comprising a conjugated modified oligonucleotide described herein has Structure A:

Lₙ-linker-MO;

wherein each L is, independently, a ligand and n is from 1 to 10; and MO is a modified oligonucoeotide.

In some embodiments, a compound has the Structure J:

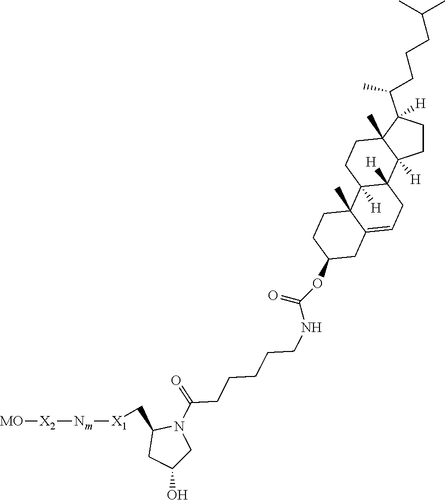

wherein each N is, independently, a modified or unmodified nucleoside and m is from 1 to 5; X₁ and X₂ are each, independently, a phosphodiester linkage or a phosphorothioate linkage; and MO is a modified oligonucleotide.

In certain embodiments, at least one of X₁ and X₂ is a phosphodiester linkage. In certain embodiments, each of X₁ and X₂ is a phosphodiester linkage.

In certain embodiments, m is 1. In certain embodiments, m is 2. In certain embodiments, m is 3, 4, or 5. In certain embodiments, m is 2, 3, 4, or 5. In certain embodiments, when m is greater than 1, each modified or unmodified nucleoside of Nₘ may be connected to adjacent modified or unmodified nucleosides of Nₘ by a phosphodiester internucleoside linkage or a phosphorothioate internucleoside linkage.

In any of the embodiments described herein, Nₘ may be N′ₚN″, where each N′ is, independently, a modified or unmodified nucleoside and p is from 0 to 4; and N″ is a nucleoside comprising an unmodified sugar moiety.

In certain embodiments, p is 0. In certain embodiments, p is 1, 2, 3, or 4. In certain embodiments, when p is 1, 2, 3, or 4, each N′ comprises an unmodified sugar moiety.

In certain embodiments, an unmodified sugar moiety is a β-D-ribose or a β-D-deoxyribose. In certain embodiments, the β-D-deoxyribose is β-D-deoxyriboadenosine.

In certain embodiments, where p is 1, 2, 3, or 4, N′ comprises a purine nucleobase. In certain embodiments, N″ comprises a purine nucleobase. In certain embodiments, a purine nucleobase is selected from adenine, guanine, hypoxanthine, xanthine, and 7-methylguanine. In certain embodiments, N′ is a β-D-deoxyriboadenosine or a β-D-deoxyriboguanosine. In certain embodiments, N″ is a β-D-deoxyriboadenosine or a β-D-deoxyriboguanosine.

In certain embodiments, p is 1, N′ and N″ are each a β-D-deoxyriboadenosine, and N′ and N″ are linked by a phosphodiester internucleoside linkage. In certain embodiments, p is 1, N′ and N″ are each a β-D-deoxyriboadenosine, and N′ and N″ are linked by a phosphodiester internucleoside linkage. In certain embodiments, p is 1, N′ and N″ are each a β-D-deoxyriboadenosine, and N′ and N″ are linked by a phosphorothioate internucleoside linkage.

In certain embodiments, where p is 1, 2, 3, or 4, N′ comprises a pyrimidine nucleobase. In certain embodiments, N″ comprises a pyrimidine nucleobase. In certain embodiments, a pyrimidine nucleobase is selected from cytosine, 5-methylcytosine, thymine, uracil, and 5,6-dihydrouracil.

In certain embodiments, the sugar moiety of each N is independently selected from a β-D-ribose, a β-D-deoxyribose, a 2′-O-methoxy sugar, a 2′-O-methyl sugar, a 2′-fluoro sugar, and a bicyclic sugar moiety. In certain embodiments, each bicyclic sugar moiety is independently selected from a cEt sugar moiety, an LNA sugar moiety, and an ENA sugar moiety. In certain embodiments, the cEt sugar moiety is an S-cEt sugar moiety. In certain embodiments, the cEt sugar moiety is an R-cEt sugar moiety.

In certain embodiments, a compound comprises a conjugate moiety linked to the 5′ terminus of the modified oligonucleotide. In certain embodiments, a compound comprises a conjugate moiety linked to the 3′ terminus of the modified oligonucleotide. In certain embodiments, a compound comprises a conjugate moiety linked to the 5′ terminus of the modified oligonucleotide. In certain embodiments, a compound comprises a first conjugate moiety linked to the 3′ terminus of the modified oligonucleotide and a second conjugate moiety linked to the 5′ terminus of the modified oligonucleotide.

### Certain Metabolic Products

Upon exposure to exonucleases and/or endonucleases in vitro or in vivo, compounds may undergo cleavage at various positions throughout the compound. The products of such cleavage may retain some degree of the activity of the parent compound, and as such are considered active metabolites. As such, a metabolic product of a compound may be used in the methods described herein. In certain embodiments, a modified oligonucleotide (unconjugated or conjugated) undergoes cleavage at the 5′ end and/or the 3′ end, resulting in a metabolic product that has 1, 2, or 3 fewer nucleotides at the 5′ end and/or the 3′ end, relative to the parent modified oligonucleotide. In certain embodiments, a modified oligonucleotide undergoes cleavage at the 5′ end, releasing the 5′-terminal nucleotide and resulting in a metabolic product that has 1 less nucleotide at the 5′ end, relative to the parent modified oligonucleotide. In certain embodiments, a modified oligonucleotide undergoes cleavage at the 5′ end, releasing two 5′-terminal nucleosides and resulting in a metabolic product that has two fewer nucleotides at the 5′ end, relative to the parent modified oligonucleotide. In certain embodiments, a modified oligonucleotide undergoes cleavage at the 3′ end, releasing the 3′-terminal nucleotide and resulting in a metabolic product that has one less nucleotide at the 3′ end, relative to the parent modified oligonucleotide. In certain embodiments, a modified oligonucleotide undergoes cleavage at the 3′ end, releasing two 3′-terminal nucleosides and resulting in a metabolic product that has two fewer nucleotides at the 3′ end, relative to the parent modified oligonucleotide.

Compounds comprising modified oligonucleotide linked to a conjugate moiety may also undergo cleavage at a site within the linker between the modified oligonucleotide and the ligand. In certain embodiments, cleavage yields the parent modified oligonucleotide comprising a portion of the conjugate moiety. In certain embodiments, cleavage yields the parent modified oligonucleotide comprising one or more subunits of the linker between the modified oligonucleotide and the ligand. For example, where a compound has the structure Lₙ-linker-X₁—Nₘ—X₂-MO, in some embodiments, cleavage yields the parent modified oligonucleotide comprising one or more nucleotides of Nₘ. In some embodiments, cleavage of a conjugated modified oligonucleotide yields the parent modified oligonucleotide. In some such embodiments, for example, where a compound has the structure Lₙ-linker-X₁—Nₘ—X₂-MO, in some embodiments, cleavage yields the parent modified oligonucleotide without any of the nucleotides of Nₘ.

### Certain Nucleobase Sequences

Any nucleobase sequences set forth herein, including but not limited to those found in the examples and in the sequence listing, are independent of any modification to the nucleic acid. As such, nucleic acids defined by a SEQ ID NO may comprise, independently, one or more modifications to one or more sugar moieties, to one or more internucleoside linkages, and/or to one or more nucleobases.

Although the sequence listing accompanying this filing identifies each nucleobase sequence as either “RNA” or “DNA” as required, in practice, those sequences may be modified with any combination of chemical modifications. One of skill in the art will readily appreciate that such designation as “RNA” or “DNA” to describe modified oligonucleotides is somewhat arbitrary. For example, a modified oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to, those in the sequence listing, are intended to encompass nucleic acids containing any combination of natural or modified RNA and/or DNA, including, but not limited to such nucleic acids having modified nucleobases. By way of further example and without limitation, a modified oligonucleotide having the nucleobase sequence “ATCGATCG” encompasses any oligonucleotide having such nucleobase sequence, whether modified or unmodified, including, but not limited to, such compounds comprising RNA bases, such as those having sequence “AUCGAUCG” and those having some DNA bases and some RNA bases such as “AUCGATCG” and oligonucleotides having other modified bases, such as “AT^{me}CGAUCG,” wherein ^{me}C indicates a 5-methylcytosine. Similarly, a modified oligonucleotide having the nucleobase sequence “AUCGAUCG” encompasses any oligonucleotide having such nucleobase sequence, whether modified or unmodified, including, but not limited to, such compounds comprising DNA bases, such as those having sequence “ATCGATCG” and those having some DNA bases and some RNA bases such as “AUCGATCG” and oligonucleotides having other modified bases, such as “AT′CGAUCG,” wherein ^{me}C indicates a 5-methylcytosine.

### Certain Synthesis Methods

Modified oligonucleotides may be made with automated, solid phase synthesis methods known in the art. During solid phase synthesis, phosphoramidite monomers are sequentially coupled to a nucleoside that is covalently linked to a solid support. This nucleoside is the 3′ terminal nucleoside of the modified oligonucleotide. Typically, the coupling cycle comprises four steps: detritylation (removal of a 5′-hydroxyl protecting group with acid), coupling (attachment of an activated phosphoroamidite to the support bound nucleoside or oligonucleotide), oxidation or sulfurization (conversion of a newly formed phosphite trimester with an oxidizing or sulfurizing agent), and capping (acetylation of unreacted 5′-hydroxyl groups). After the final coupling cycle, the solid support-bound oligonucleotide is subjected to a detritylation step, followed by a cleavage and deprotection step that simultaneously releases the oligonucleotide from the solid support and removes the protecting groups from the bases. The solid support is removed by filtration, the filtrate is concentrated and the resulting solution is tested for identity and purity. The oligonucleotide is then purified, for example using a column packed with anion-exchange resin.

GalNAc-conjugated modified oligonucleotides may be made with automated solid phase synthesis, similar to the solid phase synthesis that produced unconjugated oligonucleotides. During the synthesis of GalNAc-conjugated oligonucleotides, the phosphoramidite monomers are sequentially coupled to a GalNAc conjugate which is covalently linked to a solid support. The synthesis of GalNAc conjugates and GalNAc conjugate solid support is described, for example, in U.S. Pat. No. 8,106,022, and International Application Publication No. WO 2013/033230, each of which is herein incorporated by reference in its entirely for the description of the synthesis of carbohydrate-containing conjugates, including conjugates comprising one or more GalNAc moieties, and of the synthesis of conjugate covalently linked to solid support.

### Certain Pharmaceutical Compositions

Provided herein are pharmaceutical compositions comprising a compound provided herein, and a pharmaceutically acceptable diluent. In certain embodiments, the pharmaceutically acceptable diluent is an aqueous solution. In certain embodiments, the aqueous solution is a saline solution. As used herein, pharmaceutically acceptable diluents are understood to be sterile diluents. Suitable administration routes include, without limitation, oral administration.

In certain embodiments, provided herein are pharmaceutical compositions comprising a compound provided herein, and sodium bicarbonate.

In certain embodiments, a pharmaceutical composition is a compound provided herein which has been prepared in a suitable diluent, adjusted to pH 7.0-9.0 with acid or base during preparation, and then lyophilized under sterile conditions. The lyophilized modified oligonucleotide is subsequently reconstituted with a suitable diluent, e.g., aqueous solution, such as water or physiologically compatible buffers such as saline solution, Hanks's solution, or Ringer's solution. In certain embodiments, the pH of the pharmaceutical composition is adjusted to about 9.0 with a solution of sodium bicarbonate. The reconstituted product is administered as a subcutaneous injection or as an intravenous infusion. The lyophilized drug product may be packaged in a 2 mL Type I, clear glass vial (ammonium sulfate-treated), stoppered with a bromobutyl rubber closure and sealed with an aluminum overseal.

In certain embodiments, a pharmaceutical composition is administered in the form of a dosage unit (e.g., tablet, capsule, bolus, etc.). In some embodiments, a pharmaceutical composition comprises a compound provided herein at a dose within a range selected from 25 mg to 250 mg. In certain embodiments, such pharmaceutical compositions comprise a compound provided herein present at a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, or 250 mg.

Pharmaceutical compositions may also contain suitable stabilizers or agents that increase the solubility of the pharmaceutical agents to allow for the preparation of highly concentrated solutions.

Pharmaceutical compositions provided herein are prepared for oral administration. In certain of such embodiments, a pharmaceutical composition is formulated by combining one or more compounds comprising a modified oligonucleotide with one or more pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject.

In certain embodiments, pharmaceutical compositions for oral use are obtained by mixing a compound comprising a modified oligonucleotide and one or more solid excipients. Suitable excipients include penetration enhancers such as a fatty acid, a bile acid, a chelating agent and a non-chelating non-surfactant. In certain embodiments, a fatty acid is selected from arachidonic acid, oleic acid, lauric acid, capric acid, caprylic acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcamitine, an acylcholine and a monoglyceride or a pharmaceutically acceptable salt thereof. In certain embodiments, a bile acid is selected from cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24, 25-dihydrofusidate, sodium glycodihydrofusidate and polyoxyethylene-9-lauryl ether or a pharmaceutically acceptable salt thereof. In certain embodiments, a chelating agent is selected from EDTA, citric acid, a salicylate, N-acyl derivative of collagen, laureth-9 and an N-amino acyl derivative of a beta-diketone or a mixture thereof. In certain embodiments, a penetration enhancer comprises sodium caprate (C10) and/or sodium caprylate (C12).

Additional suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner. In certain embodiments, the pharmaceutical compositions provided herein may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents.

### Certain Kits

The present invention also provides kits. In some embodiments, the kits comprise one or more compounds provided herein. In some embodiments, a compound provided herein is present within a vial. A plurality of vials, such as 10, can be present in, for example, dispensing packs. In some embodiments, the vial is manufactured so as to be accessible with a syringe. The kit can also contain instructions for using the compounds provided herein.

In some embodiments, the kits may be used for administration of a compound provided herein to a subject. In such instances, in addition to comprising at least one compound provided herein, the kit can further comprise one or more of the following: syringe, alcohol swab, cotton ball, and/or gauze pad. In some embodiments, the compounds complementary to a microRNA can be present in a pre-filled syringe (such as a single-dose syringes with, for example, a 27 gauge, ¼ inch needle with a needle guard), rather than in a vial. A plurality of pre-filled syringes, such as 10, can be present in, for example, dispensing packs. The kit can also contain instructions for administering a compound provided herein.

### Certain Experimental Models

In certain embodiments, the present invention provides methods of using and/or testing a compound provided herein in an experimental model. Those having skill in the art are able to select and modify the protocols for such experimental models to evaluate a compound provided herein.

The effects of antisense inhibition of a microRNA following the administration of anti-miR compounds may be assessed by a variety of methods known in the art. In certain embodiments, these methods are used to quantitate microRNA levels in cells or tissues in vitro or in vivo. In certain embodiments, changes in microRNA levels are measured by microarray analysis. In certain embodiments, changes in microRNA levels are measured by one of several commercially available PCR assays, such as the TaqMan® MicroRNA Assay (Applied Biosystems, a Life Technologies brand).

In vitro activity of anti-miR compounds may be assessed using a luciferase cell culture assay. In this assay, a microRNA luciferase sensor construct is engineered to contain one or more binding sites of the microRNA of interest fused to a luciferase gene. When the microRNA binds to its cognate site in the luciferase sensor construct, luciferase expression is suppressed. When the appropriate anti-miR is introduced into the cells, it binds to the target microRNA and relieves suppression of luciferase expression. Thus, in this assay anti-miRs that are effective inhibitors of the microRNA of interest will cause an increase in luciferase expression.

Activity of anti-miR compounds may be assessed by measuring the mRNA and/or protein level of a target of a microRNA. A microRNA binds to a complementary site within one or more target RNAs, leading to suppression of a target RNA, thus inhibition of the microRNA results in the increase in the level of mRNA and/or protein of a target of the microRNA (i.e., derepression). The derepression of one or more target RNAs may be measured in vivo or in vitro. For example, a target of miR-122 is aldolase A (ALDOA). Inhibition of miR-122 results in an increase in the level of ALDOA mRNA, thus ALDOA mRNA levels may be used to evaluate the inhibitory activity of an anti-miR-122 compound.

### EXAMPLES

The following examples are presented in order to more fully illustrate some embodiments of the invention. They should, in no way be construed, however, as limiting the broad scope of the invention. Those of ordinary skill in the art will readily adopt the underlying principles of this discovery to design various compounds without departing from the spirit of the current invention.

### Example 1

Oral administration of oligonucleotides has several features that may be advantageous to subcutaneous administration, for example, improved patient compliance, improved convenience of dosing, and lack of subcutaneous injection site reactions. To date, oral administration of anti-miR compounds has not been well characterized. Accordingly, oral administration of anti-miR compounds was evaluated in experimental animal models.

Table 1 illustrates the sequence and sugar moieties of unconjugated modified oligonucleotides complementary to microRNAs. RG5116 is 100% complementary to nucleotides 1-19 of let-7. RG5365 is 100% complementary to nucleotides 1-9 of let-7. RG7443 is 100% complementary to nucleotides 2-10 of miR-122. Nucleosides followed by subscript “D” are β-deoxyribonucleotides, nucleosides followed by subscript “M” are 2′-O-methyl nucleosides, nucleosides followed by subscript “E” are 2′-O-methoxyethyl nucleosides, nucleosides followed by subscript “F” are 2′-fluoro nucleosides, and nucleosides followed by subscript “K” are S-cEt nucleosides. Except as otherwise indicated, all internucleoside linkages are phosphorothioate linkages and all cytosines are non-methylated cytosines.

TABLE 1
Unconjugated Modified Oligonucleotides

| | | | | | | | | | | | | | | | | | | | | SEQ |
|---|---|---|---|---|---|---|---|---|---|---|---|---|---|---|---|---|---|---|---|---|
| Compound | | | | | | | | | | | | | | | | | | | | ID |
| No. | Sequence (5′ to 3′) and Sugar Moieties | | | | | | | | | | | | | | | | | | | NO |
| RG5116 | T_{E} | A_{K} | U_{K} | A_{E} | C_{K} | A_{E} | A_{E} | C_{K} | C_{M} | U_{M} | A_{K} | C_{K} | U_{M} | A_{K} | C_{M} | C_{K} | U_{M} | C_{K} | A_{K} | 1 |
| RG5365 | | | | | | | | | | | A_{K} | C_{K} | U_{M} | A_{F} | C_{F} | C_{F} | U_{M} | C_{K} | A_{K} | |
| RG7443 | | | | | | | | | | | U_{K} | C_{K} | A_{D} | C_{K} | A_{D} | C_{K} | T_{D} | C_{K} | C_{K} | |
| RG4326 | | | | | | | | | | | A_{K} | G_{K} | C_{M} | A_{F} | C_{F} | U_{F} | U_{M} | U_{K} | G_{K} | |

Table 2 illustrates the structure of compounds comprising a modified oligonucleotide and a conjugate moiety.

TABLE 2
Conjugated Modified Oligonucleotides

| | | Modified | |
|---|---|---|---|
| Compound | Conjugate | Oligonucleotide | |
| No. | Type | (MO) | Conjugate Moiety |
| RG5136 | cholesterol | RG5116 | Structure J, where |
| | | | X₂ is a phophodiester linkage, |
| | | | m is 1, |
| | | | Nₘ is a β-D-deoxynucleoside (dA), |
| | | | X₁ is a phosphodiester linkage |
| RG6650 | triantennary | RG7443 | Structure C, where |
| | GalNAc | | X₂ is a phophodiester linkage, |
| | | | m is 1, |
| | | | Nₘ is a β-D-deoxynucleoside (dA), |
| | | | X₁ is a phosphodiester linkage |

Structure C: 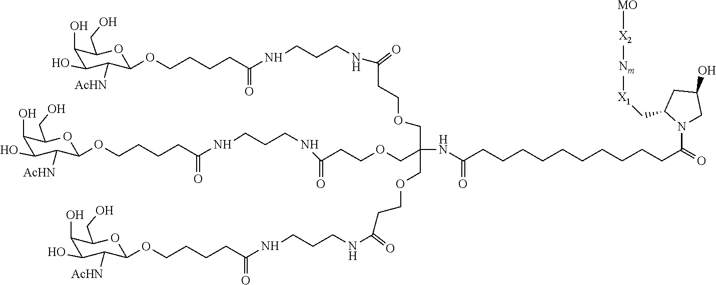
Structure J:

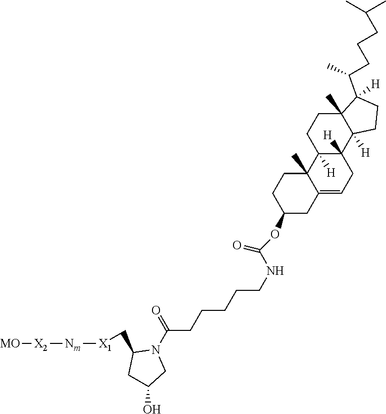

### Pharmacodynamic Activity of Anti-Let-7 Compounds

The pharmacodynamic (PD) activity of the anti-let-7 compounds was evaluated using a multi-gene PD signature. Following treatment with anti-miR, the change in expression of 14 to 18 validated let-7 target genes is measured by RT-PCR. The log 2 of the fold changes of the selected let-7 target genes is averaged and the average is considered the let-7 PD signature score (let-7 PD sig), which is used as the indicator of pharmacodynamic activity.

Groups of five mice each were treated with the anti-let-7 compounds. Dosages and administration routes are indicated in Table 3, where “s.c.” indicates subcutaneous administration and “p.o.” indicates oral administration. Anti-miR compounds were delivered in PBS for subcutaneous administration. Orally administered anti-miR compounds were prepared in a solution of PBS and 0.3 M sodium bicarbonate (BC) was added to a pH of 9.5. For oral dosing, animals were fasted for 12 hours prior. In all treatment groups, mice were administered a single dose and sacrificed four days later. Liver, kidney and colon tissues were collected for pharmacodynamic and pharmacokinetic analyses. RNA was isolated from each tissue and the mean let-7 PD signature scores for each group were determined.

TABLE 3
let-7 PD Sig Scores Following anti-miR Treatment

| | | | | | Liver | | Kidney | | Colon | |
|---|---|---|---|---|---|---|---|---|---|---|
| | | | | | Mean | | Mean | | Mean | |
| | Cmpnd | | Dose | | Let-7 | Std | Let-7 | Std | Let-7 | Std |
| Group | Type | Treatment | mg/kg | Route | PD sig | Dev | PD sig | Dev | PD sig | Dev |
| 1 | | PBS | 0 | s.c. | 0.0000 | 0.0839 | 0.0000 | 0.1334 | 0.0000 | 0.1176 |
| 2 | 19-mer | RG5116 | 30 | s.c. | 0.4360 | 0.0854 | 0.5224 | 0.0646 | 0.0934 | 0.1153 |
| | | in PBS | | | | | | | | |
| 3 | 9-mer | RG5365 | 30 | s.c. | 0.6036 | 0.1214 | 0.6038 | 0.0763 | 0.2113 | 0.0658 |
| | | in PBS | | | | | | | | |
| 4 | | BC | 0 | p.o. | 0.0000 | 0.0425 | 0.0000 | 0.0994 | 0.0000 | 0.1190 |
| 5 | 19-mer | RG5116 | 100 | p.o. | 0.1886 | 0.3302 | 0.0021 | 0.0874 | 0.0307 | 0.0921 |
| | | in BC | | | | | | | | |
| 6 | 9-mer | RG5365 | 100 | p.o. | 0.3572 | 0.1885 | 0.5480 | 0.0996 | 0.4772 | 0.2736 |
| | | in BC | | | | | | | | |
| 7 | 19-mer + | RG5136 | 100 | p.o. | 0.1786 | 0.1030 | −0.0642 | 0.1031 | 0.0767 | 0.0455 |
| | chol. | in BC | | | | | | | | |

As shown in Table 3, in the kidney, the PD activity of 100 mg/kg of the 9-mer administered orally is comparable to the PD activity of 30 mg/kg the 9-mer administered subcutaneously. In the liver, while the PD activity of the 9-mer is greater following subcutaneous administration, the 9-mer does exhibit a PD effect when administered orally. Notably, the robust PD effects following oral administration are observed for the shorter 9-mer compound, but not the 19-mer or cholesterol-conjugated 19-mer.

### Pharmacodynamic Activity of Anti-Let-7 Compounds-Dose Response

To further evaluate the PD activity of orally administered anti-miRs, an additional experiment was performed, with doses of RG5365 ranging from 3 to 100 mg/kg.

Groups of five mice each were treated with the anti-let-7 compounds as shown in Table 4, where “s.c.” indicates subcutaneous administration and “p.o.” indicates oral administration. Anti-miR compounds were delivered in PBS for subcutaneous administration. Orally administered anti-miR compounds were prepared in a solution of PBS and 0.3 M sodium bicarbonate (BC) was added to a pH of 9.5. For oral dosing, animals were fasted for 12 hours prior. For all treatment groups, mice were administered a single dose and sacrificed four days later. Liver, kidney and colon tissues was collected for pharmacodynamic and pharmacokinetic analyses. RNA was isolated from each tissue and the mean let-7 PD signature scores for each group were determined.

TABLE 4
let-7 PD Sig Scores Following anti-miR Treatment

| | | | | | Liver | | Kidney | | Colon | |
|---|---|---|---|---|---|---|---|---|---|---|
| | | | | | Mean | | Mean | | Mean | |
| | Cmpnd | | Dose | | Let-7 | Std | Let-7 | Std | Let-7 | Std |
| Group | Type | Treatment | mg/kg | Route | PD sig | Dev | PD sig | Dev | PD sig | Dev |
| 1 | | PBS | 0 | s.c. | 0.0000 | 0.0487 | 0.0000 | 0.3206 | 0.0000 | 0.1146 |
| 2 | 19-mer | RG5116 | 30 | s.c. | 0.4816 | 0.0490 | 0.9445 | 0.1431 | 0.2167 | 0.3978 |
| | | in PBS | | | | | | | | |
| 3 | 9-mer | RG5365 | 30 | s.c. | 0.9629 | 0.0567 | 1.2670 | 0.1043 | 0.2886 | 0.0965 |
| | | in PBS | | | | | | | | |
| 4 | | PBS | 0 | p.o. | 0.0000 | 0.0707 | 0.0000 | 0.2667 | −0.1309 | 0.9074 |
| 5 | 19-mer | RG5116 | 100 | p.o. | −0.0649 | 0.0360 | 0.1212 | 0.1812 | 0.3586 | 0.0754 |
| | | in PBS | | | | | | | | |
| 6 | 9-mer | RG5365 | 100 | p.o. | 0.2038 | 0.1099 | 0.8247 | 0.4013 | 0.8891 | 0.3072 |
| | | in PBS | | | | | | | | |
| 7 | | BC | 0 | p.o. | 0.0000 | 0.0784 | 0.0000 | 0.2766 | 0.0000 | 0.1423 |
| 8 | 19-mer | RG5116 | 100 | p.o. | −0.0573 | 0.3619 | 0.1088 | 0.1163 | −0.0039 | 0.2126 |
| | | in BC | | | | | | | | |
| 9 | 9-mer | RG5365 | 3 | p.o. | 0.0885 | 0.0688 | 0.2068 | 0.2357 | −0.2953 | 0.1398 |
| | | in BC | | | | | | | | |
| 10 | 9-mer | RG5365 | 10 | p.o. | 0.0656 | 0.1074 | 0.4343 | 0.1135 | −0.1841 | 0.2962 |
| | | in BC | | | | | | | | |
| 11 | 9-mer | RG5365 | 30 | p.o. | 0.0627 | 0.0995 | 0.5871 | 0.2890 | 0.0761 | 0.2708 |
| | | in BC | | | | | | | | |
| 12 | 9-mer | RG5365 | 100 | p.o. | 0.4987 | 0.1715 | 0.8738 | 0.1338 | 0.2522 | 0.4399 |
| | | in BC | | | | | | | | |

Also evaluated in this experiment was the amount of anti-miR present in liver and kidney following treatment.

Tissue concentrations of anti-miR compound was measured by extraction of compound using protein digestion in a lysis buffer, followed by hybridizing the anti-miR compound to a fluorescent probe that has sequence complementarity to the compound. After hybridization, the identity and concentration of the compound was assessed using high performance liquid chromatography coupled with fluorescence (HPLC-FL).

TABLE 5
Anti-miR Concentration in Tissues

| | Com- | | | | Liver | | Kidney | |
|---|---|---|---|---|---|---|---|---|
| | pound | Treat- | Dose | | Mean | Std | Mean | Std |
| Group | Type | ment | mg/kg | Route | ug/g | Dev | ug/g | Dev |
| 1 | | PBS | 0 | s.c. | | | | |
| 2 | 19-mer | RG5116 | 30 | s.c. | 45.94 | 5.61 | 158.82 | 12.81 |
| | | in PBS | | | | | | |
| 3 | 9-mer | RG5365 | 30 | s.c. | <7.8 | | 18.96 | 4.65 |
| | | in PBS | | | | | | |
| 4 | | PBS | 0 | p.o. | | | | |
| 5 | 19-mer | RG5116 | 100 | p.o. | 0.41 | 0.09 | 1.29 | 0.36 |
| | | in PBS | | | | | | |
| 6 | 9-mer | RG5365 | 100 | p.o. | <0.4 | | 1.31 | 0.35 |
| | | in PBS | | | | | | |
| 7 | | BC | 0 | p.o. | | | | |
| 8 | 19-mer | RG5116 | 100 | p.o. | 0.51 | 0.37 | 1.85 | 1.45 |
| | | in BC | | | | | | |
| 9 | 9-mer | RG5365 | 100 | p.o. | <0.4 | | 1.56 | 0.49 |
| | | in BC | | | | | | |
| 10 | 9-mer | RG5365 | 30 | p.o. | <0.4 | | 0.34 | 0.03 |
| | | in BC | | | | | | |
| 11 | 9-mer | RG5365 | 10 | p.o. | <0.4 | | 0.05 | 0.1 |
| | | in BC | | | | | | |
| 12 | 9-mer | RG5365 | 3 | p.o. | <0.4 | | <0.2 | |
| | | in BC | | | | | | |

In this experiment, following oral administration, the 9-mer exhibited a statistically significant, dose-dependent PD effect in the kidney. While a statistically significant PD effect was observed with the highest dose of the 9-mer in the liver, a PD effect was not observed with the three lowest doses. As with the previous experiment, following oral administration, the PD effects in the liver and kidney were observed with the 9-mer, but not the 19-mer. Interestingly, while the amount of anti-miR detected in kidney following oral administration of the 9-mer was less than that detected following subcutaneous administration, the PD effect was substantial. This observation suggests that following oral administration, a robust PD effect can be achieved with the kidney being exposed to a relatively small amount of anti-miR compound.

### Pharmacodynamic Activity of Anti-miR-122 Compounds

An experiment was performed to evaluate the PD effects of anti-miRs targeted to an additional microRNA, miR-122. RG5365, targeted to let-7, was included for comparison to other experiments described herein.

Groups of five mice each were treated with the anti-let-7 compounds and anti-miR-122 compounds as shown in Table 6, where “s.c.” indicates subcutaneous administration and “p.o.” indicates oral administration. Anti-miR compounds were delivered in PBS for subcutaneous administration. Orally administered anti-miR compounds were prepared in a solution of PBS and 0.3 M sodium bicarbonate (BC) was added to a pH of 9.5. For oral dosing, animals were fasted for 12 hours prior. For all treatment groups, mice were administered a single dose and sacrificed four days later. Liver and kidney tissue was collected for pharmacodynamic and pharmacokinetic analyses. RNA was isolated from liver and kidney.

For the anti-let-7 compound, a let-7 PD signature score was generated for both liver and kidney tissue. For anti-miR-122 compounds, ALDOA depression in the liver and kidney was measured by RT-PCR.

TABLE 6
PD Effects Following Anti-miR Treatment

| | Compound | | Dose | | Liver | | Kidney | |
|---|---|---|---|---|---|---|---|---|
| Group | Type | Treatment | mg/kg | Route | PD Sig | Std Dev | PD Sig | Std Dev |
| 1 | | PBS | 0 | s.c. | 0.0000 | 0.1029 | | |
| 2 | 9-mer + GalNAc | RG6650 | 30 | s.c. | 1.704 | 0.3447 | 0.1353 | 0.3123 |
| | | in PBS | | | | | | |
| 3 | 9-mer | RG7443 | 30 | s.c. | 1.945 | 0.3543 | −0.0515 | 0.0792 |
| | | in PBS | | | | | | |
| 4 | 9-mer | RG5365 | 30 | s.c. | 0.8224 | 0.1352 | 1.214 | 0.0239 |
| | | in PBS | | | | | | |
| 5 | 9-mer | BC | 0 | p.o. | 0.0000 | 0.0941 | | |
| 6 | 9-mer + GalNAc | RG6650 | 100 | p.o. | 1.84 | 0.3249 | −0.01459 | 0.1519 |
| | | in BC | | | | | | |
| 7 | 9-mer | RG7443 | 100 | p.o | 0.3448 | 0.2453 | −0.00279 | 0.1407 |
| | | in BC | | | | | | |
| 8 | 9-mer | RG5365 | 100 | p.o. | 0.2307 | 0.0455 | 0.7742 | 0.0597 |
| | | in BC | | | | | | |

TABLE 7
Anti-miR Amounts in Kidney and Liver Tissues

| | | | | | Liver | | Kidney | |
|---|---|---|---|---|---|---|---|---|
| | Compound | | Dose | | Mean | Std | Mean | Std |
| Group | Type | Treatment | mg/kg | Route | ug/g | Dev | ug/g | Dev |
| 1 | | PBS | 0 | s.c. | | | | |
| 2 | 9-mer + GalNAc | RG6650 in PBS | 30 | s.c. | 13.94 | 1.357 | 12.91 | 1.566 |
| 3 | 9-mer | RG7443 in PBS | 30 | s.c. | 1.48 | 0.239 | 21.8 | 1.485 |
| 4 | 9-mer | RG5365 in PBS | 30 | s.c. | 1.44 | 0.1657 | 24.88 | 3.257 |
| 5 | 9-mer | BC | 0 | p.o. | | | | |
| 6 | 9-mer + GalNAc | RG6650 in BC | 100 | p.o. | 0.54* | n.d. | ≤0.4 | |
| 7 | 9-mer | RG7443 in BC | 100 | p.o. | ≤0.4 | | 1.392 | 0.2398 |
| 8 | 9-mer | RG5365 in BC | 100 | p.o. | ≤0.4 | | 0.726 | 0.09607 |

For RG6650 in liver (*), results in all but one animal were non-quantifiable, thus a mean ug/g is not reported.

In this experiment, following oral administration, the 9-mer targeted to let-7 exhibited a statistically significant, PD effect in the kidney and liver. A PD effect following treatment with anti-miR-122 compounds was not observed, however, derepression of ALDOA via miR-122 inhibition may not be the optimal indicator of PD activity in kidney. Notably, while oral administration of the unconjugated anti-miR-122 compound RG7443 did not produce a substantial PD effect in the liver, oral administration of the GalNAc-conjugated compound RG6650 yielded a PD effect comparable to that observed following subcutaneous administration.

### Pharmacodynamic Activity of Anti-Let-7 and Anti-miR-17 Compounds

An experiment was performed to evaluate the PD effects of anti-miRs targeted to an additional microRNA, miR-17. RG5365, targeted to let-7, was included for comparison to other experiments described herein.

Groups of 5 mice each were treated with a single dose of anti-miR compound as shown in Table 5. Animals were fasted for 12 hours prior to oral administration. Animals were sacrificed four days following dosing. Kidney tissue was collected for pharmacodynamic and pharmacokinetic analyses.

The PD effects of the compounds were determined by using the microRNA polysome shift assay (miPSA), which provides a direct measurement of microRNA target engagement by a complementary anti-miR (Androsavich et al., Nucleic Acids Research, 2015, 44: e 13). This assay was used to determine the extent to which anti-miR compounds directly engage their microRNA target in the kidney in normal and PKD mice. The miPSA relies on the principle that active miRNAs bind to their mRNA targets in translationally active high molecular weight (HMW) polysomes, whereas the inhibited miRNAs reside in the low MW (LMW) polysomes. Treatment with anti-miR results in a shift of the microRNA from HMW polysomes to LMW polysomes, i.e. displacement of the microRNA from the HMW polysomes to LMW polysomes. From the miPSA assay, a measure of displacement, or the PSA Score, is determined.

Shown in Table 8 are the mean PSA scores in kidney tissue, and the mean amounts of anti-miR found in the kidney tissue.

TABLE 8
PSA Scores Following Anti-miR Treatment

| | Com- | | | | | | | |
|---|---|---|---|---|---|---|---|---|
| | pound | | Dose | | PSA | Std | Mean | Std |
| Group | Type | Treatment | mg/kg | Route | Score | Dev | ug/g | Dev |
| 1 | | PBS | 0 | s.c. | .005 | 0.6168 | | |
| 2 | 9- | RGLS4326 | 30 | s.c. | 3.464 | 0.2105 | 32.4 | 3.03 |
| | mer | in PBS | | | | | | |
| 3 | 9- | RG5365 | 30 | s.c. | 2.906 | 0.7856 | 26.37 | 5.14 |
| | mer | in PBS | | | | | | |
| 4 | | BC | 0 | p.o. | 0 | 0.2818 | | |
| 5 | 9- | RGLS4326 | 100 | p.o. | 1.432 | 0.402 | 0.66 | 0.15 |
| | mer | in PBS | | | | | | |
| 6 | 9- | RG5365 | 100 | p.o. | 1.615 | 0.5211 | 1.05 | 0.34 |
| | mer | in PBS | | | | | | |

As shown in Table 8, as measured by the PSA displacement score, each 9-mer anti-miR exhibited a significant PD effect in the kidney. Interestingly, while the amount of anti-miR detected in kidney following oral administration of each 9-mer was less than that detected following subcutaneous administration, the PD effect was substantial. This observation suggests that following oral administration, a substantial PD effect can be achieved with the kidney being exposed to a lower amount of anti-miR than following subcutaneous administration.

Collectively, these experiments demonstrated robust PD effects of orally administered conjugated and unconjugated short anti-miR compounds in the target tissue (liver for conjugated anti-miRs and kidney for unconjugated anti-miRs), despite a relatively low tissue level detected.

### Example 2: Additional Anti-miR-122 Compounds

To further evaluate the relative activities of compounds delivered orally and subcutaneously, additional anti-miR-122 compounds of varying lengths, conjugate moieties and internucleoside linkages were tested. The compounds are shown in Table 9. Compounds conjugated to a GalNAc moiety have a structure as shown in Structure C in Example 1, where X₂ is a phophodiester linkage, m is 1, Nₘ is a β-D-deoxynucleoside (dA), X₁ is a phosphodiester linkage, and MO is the modified oligonucleotide. A compound conjugated to a cholesterol moiety has a structure as shown in Structure J in Example 1, where X₂ is a phophodiester linkage, m is 1, Nₘ is a β-D-deoxynucleoside (dA), X₁ is a phosphodiester linkage, and MO is the modified oligonucleotide.

In this set of compounds, the compound comprising the longest oligonucleotide tested was the 18-mer RG2459, also known as RG-101. A series of truncations of RG2459 were generated, producing compounds RG7441, RG3396, RG7442, and RG6650, having modified oligonucleotides of 15, 14, 13, and 9 linked nucleosides, respectively. Each of these four compounds comprises a GalNAc moiety conjugated to the modified oligonucleotide via a phosphodiester-linked β-D-deoxyriboadenosine.

RG6386 is similar to RG2459, with changes to the sugar modifications at the 5′ end of the modified oligonucleotide. RG8210 comprises a modified oligonucleotide with the same chemical modifications of RG6650 but is instead conjugated to a cholesterol moiety via a phosphodiester-linked via a β-D-deoxyriboadenosine. RG7443 has the same chemical modification pattern as the modified oligonucleotide of RG6650 and RG8120 but is unconjugated. RG2634 and RG3059 both comprise uniformly cEt-modified modified oligonucleotides, conjugated to a GalNAc moiety via a phosphodiester-linked β-D-deoxyriboadenosine. Whereas the internucleoside linkages of the modified oligonucleotide of RG2634 as phosphorothioate, the internucleoside linkages of RG3059 are phosphodiester. The nucleobase sequence of the modified oligonucleotide of RG4474 comprises mismatches to the sequence of miR-122 and was used as a mismatch control compound.

TABLE 9
Anti-miR-122 Compounds

| Com- | | | | | | | | | | | | | | | | | | | | | SEQ |
|---|---|---|---|---|---|---|---|---|---|---|---|---|---|---|---|---|---|---|---|---|---|
| pound | Modified Oligonucleotide (MO) | | | | | | | | | | | | | | | | | | Conjugate | MO | ID |
| Number | Nuclcobase Sequence (5′ to 3′) and Chemistry | | | | | | | | | | | | | | | | | | Moiety | Length | NO: |
| RG2459 | A_{E} | ^{Me}C_{E} | A_{E} | ^{Me}C_{E} | ^{Me}C_{E} | A_{E} | T_{E} | T_{D} | G_{D} | U_{K} | C_{K} | A_{D} | C_{K} | A_{D} | C_{K} | T_{D} | C_{K} | C_{K} | GalNAc | 18 | 2 |
| RG6386 | | | A_{E} | C_{K} | C_{K} | A_{E} | U_{K} | T_{D} | G_{D} | U_{K} | C_{K} | A_{D} | C_{K} | A_{D} | C_{K} | T_{D} | C_{K} | C_{K} | GalNAc | 16 | 3 |
| RG7441 | | | | | ^{M}ₑC_{E} | A_{E} | T_{E} | T_{D} | G_{D} | U_{K} | C_{K} | A_{D} | C_{K} | A_{D} | C_{K} | T_{D} | C_{K} | C_{K} | GalNAc | 14 | 4 |
| RG3396 | | | | | | A_{E} | T_{E} | T_{D} | G_{D} | U_{K} | C_{K} | A_{D} | C_{K} | A_{D} | C_{K} | T_{D} | C_{K} | C_{K} | GalNAc | 13 | 5 |
| RG7442 | | | | | | | T_{E} | T_{D} | G_{D} | U_{K} | C_{K} | A_{D} | C_{K} | A_{D} | C_{K} | T_{D} | C_{K} | C_{K} | GalNAc | 12 | 6 |
| RG6650 | | | | | | | | | | U_{K} | C_{K} | A_{D} | C_{K} | A_{D} | C_{K} | T_{D} | C_{K} | C_{K} | GalNAc | 9 | |
| RG8120 | | | | | | | | | | U_{K} | C_{K} | A_{D} | C_{K} | A_{D} | C_{K} | T_{D} | C_{K} | C_{K} | Cholesterol | 9 | |
| RG7443 | | | | | | | | | | U_{K} | C_{K} | A_{D} | C_{K} | A_{D} | C_{K} | T_{D} | C_{K} | C_{K} | None | 9 | |
| RG2634 | | | | | | | | | | | C_{K} | A_{K} | C_{K} | A_{K} | C_{K} | T_{K} | C_{K} | C_{K} | GalNAc | 8 | |
| RG3059 | | | | | | | | | | | C_{K} | ^{O}A_{K} | ^{O}C_{K} | ^{O}A_{K} | ^{O}C_{K} | ^{O}T_{K} | ^{O}C_{K} | ^{O}C_{K} | GalNAc | 8 | |
| RG4474 | A_{E} | A_{E} | ^{Me}C_{E} | A_{E} | ^{Me}C_{E} | C_{E} | T_{E} | T_{D} | G_{D} | U_{K} | C_{K} | A_{D} | C_{K} | A_{D} | C_{K} | T_{D} | C_{K} | C_{K} | GalNAc | 18 | 7 |

Nucleosides followed by a subscript “D” indicate β-D-deoxyribonucleosides; nucleosides followed by a subscript “E” indicate 2′-MOE nucleosides; nucleosides followed by a subscript “S” indicate S-cEt nucleosides. Phosphodiester internucleoside linkages are indicated by a superscript “0”; all other internucleoside linkages are phosphorothioate. “Me” indicates a 5-methyl group on the base of the nucleoside.

Compounds RG2459, RG6650, RG6386, RG7441, RG2634, and RG7442 were tested in a first experiment. Compounds RG7443, RG6650, RG4474, RG3059, RG3396, and RG8120 were tested in a second experiment. Groups of three mice each were administered 100 mg/kg dose orally (p.o.) or a 10 mg/kg dose subcutaneously (s.c.). Anti-miR compounds were delivered in PBS for subcutaneous administration. Orally administered anti-miR compounds were prepared in a solution of PBS and 0.3 M sodium bicarbonate (BC) was added to a pH of 9.5. For oral dosing, animals were fasted for 12 hours prior. For all treatment groups, mice were administered a single dose and sacrificed two, four, or seven days later. Liver tissue was collected for pharmacodynamic and pharmacokinetic analyses. RNA was isolated from liver and ALDOA depression in the liver was measured via RT-PCR and normalized to the housekeeping gene RplpO, also measured by RT-PCR.

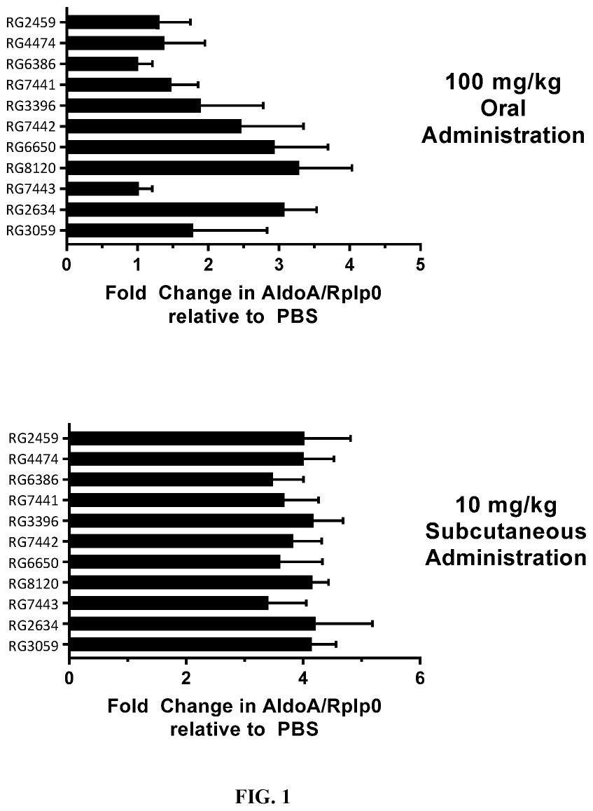

As the de-repression of ALDOA was generally similar across study end days 2, 4 and 7, the mean ALDOA de-repression for each treatment across days 2, 4, and 7 was calculated and is shown in FIG. 1.

When administered subcutaneously, the compounds exhibited similar activities. Differences in compound activity were apparent, however, following oral administration. Notably, following oral administration, compounds having shorter anti-miR lengths exhibited a trend towards greater activity. For example, RG2459, RG6386, and RG7441 which comprise anti-miRs of 18, 16, and 14 linked nucleosides did not produce a substantial de-repression of ALDOA, relative to PBS treatment. Further, the unconjugated 9-mer RG7443 and the conjugated compound RG3059 having a phosphodiester linked 8-mer did not substantially de-repress ALDOA. The negative control RG4474 did not de-repress ALDOA. RG7442, which comprises a 12-mer anti-miR, and RG3396, which comprises a 13-mer anti-miR, were more active. The GalNAc-conjugated compounds RG6650 and RG2634, which comprise a 9-mer and 8-mer anti-miR, respectively, exhibited de-repression of ALDOA. Similarly, the cholesterol-conjugated RG8120 which comprises a 9-mer anti-miR, exhibited de-repression of ALDOA. Thus, the activity of conjugated compounds increases as the length of the anti-miR decreases, demonstrating that shorter-length conjugated compounds are more active following oral administration.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, GENBANK® accession numbers, and the like) cited in the present application is specifically incorporated herein by reference in its entirety.

What is claimed:

1. A method of inhibiting the activity of a microRNA, comprising administering to a subject a compound comprising a modified oligonucleotide complementary to the microRNA, or a pharmaceutically acceptable salt thereof, wherein the modified oligonucleotide has a length of 6 to 25 linked nucleotides and wherein the administration is oral administration.

2. The method of claim 1, wherein the modified oligonucleotide is fully complementary to the microRNA.

3. The method of claim 1 or 2, wherein the microRNA is expressed in the kidney and the compound consists of the modified oligonucleotide.

4. The method of claim 1 or 2, wherein the microRNA is expressed in the liver and the compound comprises the modified oligonucleotide linked to a conjugate moiety.

5. The method of any one of claims 1 to 4, wherein the modified oligonucleotide is 8 to 13 linked nucleosides in length, or 8 to 12 linked nucleotides in length.

6. The method of any one of claims 1 to 4, wherein the modified oligonucleotide is 8 linked nucleosides in length.

7. The method of any one of claims 1 to 4, wherein the modified oligonucleotide is 9 linked nucleosides in length.

8. The method of any one of claims 1 to 4, wherein the modified oligonucleotide is 10 linked nucleosides in length.

9. The method of any one of claims 1 to 4, wherein the modified oligonucleotide is 11 linked nucleosides in length.

10. The method of any one of claims 1 to 4, wherein the modified oligonucleotide is 12 linked nucleosides in length.

11. The method of any one of claims 1 to 4, wherein the modified oligonucleotide is 13 linked nucleosides in length.

12. The method of any one of claims 1 to 11, wherein the modified oligonucleotide comprises at least one nucleoside with a modified sugar moiety.

13. The method of claim 12, wherein each nucleoside of the modified oligonucleotide comprises a modified sugar moiety.

14. The method of claim 12 or 13, wherein each modified sugar moiety is independently selected from a 2′-O-methyl sugar moiety, a 2′-O-methoxyethyl sugar moiety, a 2′-fluoro sugar moiety, and a bicyclic sugar moiety.

15. The method of claim 14, wherein each bicyclic sugar moiety is independently selected from a cEt sugar moiety and an LNA sugar moiety.

16. The method of claim 15, wherein the cEt nucleoside is an S-cEt nucleoside.

17. The method of any of claims 1 to 11, wherein the modified oligonucleotide comprises a plurality of non-bicyclic nucleosides and a plurality of bicyclic nucleosides.

18. The method of claim 17, wherein each non-bicyclic nucleoside is independently selected from a 2′-O-methyl nucleoside, a 2′-O-methoxyethyl nucleoside, and a 2′-fluoronucleoside.

19. The method of claim 18, wherein each bicyclic nucleoside is selected from a cEt nucleoside and an LNA nucleoside.

20. The method of claim 19, wherein the cEt nucleoside is an S-cEt nucleoside.

21. The method of any one of claims 1 to 20, wherein the modified oligonucleotide comprises at least one modified internucleoside linkage.

22. The method of any one of claims 1 to 20, wherein each internucleoside linkage of the modified oligonucleotide is a modified internucleoside linkage.

23. The method of claim 21 or 22, wherein the modified internucleoside linkage is a phosphorothioate linkage.

24. The method of claim 4, wherein the conjugate moiety comprises a cholesterol moiety or a carbohydrate moiety.

25. The method of claim 24, wherein the carbohydrate moiety is selected from is selected from N-acetylgalactosamine, galactose, galactosamine, N-formylgalactosamine, N-propionyl-galactosamine, N-n-butanoylgalactosamine, and N-iso-butanoyl-galactosamine.

26. The method of claim 4, wherein the compound has the structure:
Lₙ-linker-X₁—Nₘ—X₂-MO;
wherein each L is, independently, a ligand and n is from 1 to 10; each N is, independently, a modified or unmodified nucleoside and m is from 1 to 5; X₁ is a phosphodiester linkage or a phosphorothioate linkage; X₂ is a phosphodiester linkage or a phosphorothioate linkage; and MO is the modified oligonucleotide.

27. The method of claim 26 comprising the structure: 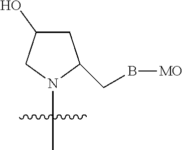
wherein:
B is selected from —O—, —S—, —N(R^{N})—, —Z—P(Z′)(Z″)O—, —Z—P(Z′)(Z″)O—Nₘ—X₁—, and —Z—P(Z′)(Z″)O—Nₘ—X₂—;
MO is the modified oligonucleotide;
R^{N} is selected from H, methyl, ethyl, propyl, isopropyl, butyl, and benzyl;
Z, Z′, and Z″ are each independently selected from O and S;
each N is, independently, a modified or unmodified nucleoside;
m is from 1 to 5;
X₁ is selected from a phosphodiester linkage and a phosphorothioate linkage;
X₂ is a phosphodiester linkage; and
the wavy line indicates the connection to the rest of the linker and ligand(s).

28. The method of claim 26 or 27, wherein n is from 1 to 5, 1 to 4, 1 to 3, or 1 to 2.

29. The method of claim 26 or 27, wherein n is 3 and each ligand is N-acetylgalactosamine.

30. The method of claim 26 or 27, wherein n is 1 and the ligand is cholesterol.

31. The method of claim 26 or 27, wherein the compound has the structure: 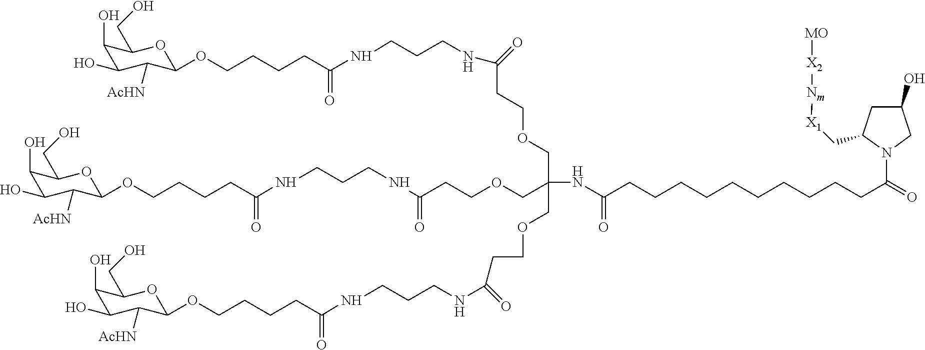
wherein each N is, independently, a modified or unmodified nucleoside and m is from 1 to 5; X₁ and X₂ are each, independently, a phosphodiester linkage or a phosphorothioate linkage; and MO is the modified oligonucleotide.

32. The method of claim 26 or 27, wherein the compound has the structure: 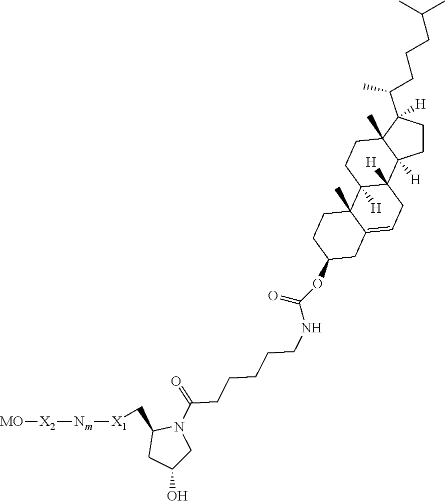
wherein each N is, independently, a modified or unmodified nucleoside and m is from 1 to 5; X₁ and X₂ are each, independently, a phosphodiester linkage or a phosphorothioate linkage; and MO is the modified oligonucleotide.

33. The method of claim 31 or 32, wherein at least one of X₁ and X₂ is a phosphodiester linkage.

34. The method of claim 31 or 32, wherein each of X₁ and X₂ is a phosphodiester linkage.

35. The method of any one of claims 26 to 34, wherein m is 1.

36. The method of any one of claims 26 to 34, wherein m is 2, 3, 4, or 5.

37. The method of any one of claims 26 to 36, wherein Nₘ is N′ₚN″, wherein each N′ is, independently, an unmodified nucleoside and p is from 0 to 4; and N″ is a nucleoside comprising an unmodified sugar moiety.

38. The method of claim 37, wherein p is 0.

39. The method of claim 37 or 38, wherein the unmodified sugar moiety is a β-D-ribose or a β-D-deoxyribose.

40. The method of claim 39, wherein the β-D-deoxyribose is β-D-deoxyriboadenosine.

41. The method of any one of claims 1 to 40, wherein the compound is present in a pharmaceutical composition.

42. The method of claim 41, wherein the pharmaceutical composition comprises a pharmaceutically acceptable diluent.

43. The method of claim 42, wherein the pharmaceutically acceptable diluent is an aqueous solution.

44. The method of claim 43, wherein the aqueous solution is a saline solution.

45. The method of claim 43 or 44, wherein the aqueous solution comprises sodium bicarbonate.
